(19) United States
(12) Patent Application Publication
(10) Pub. No.: US 2026/0069877 A1
(43) Pub. Date: Mar. 12, 2026

(54) SHORT INTERVAL HEART RATE VISUALIZATION

(71) Applicant: ZOLL Medical Corporation, Chelmsford, MA (US)

(72) Inventor: Michael A. Sipe, Pittsburgh, PA (US)

(73) Assignee: ZOLL Medical Corporation, Chelmsford, MA (US)

(21) Appl. No.: 19/323,250

(22) Filed: Sep. 9, 2025

Related U.S. Application Data
(60) Provisional application No. 63/693,898, filed on Sep. 12, 2024.

(51) Int. Cl. A61N 1/39; G06T 11/20

(52) U.S. Cl. CPC A61N 1/3993; A61N 1/3904; A61N 1/3925; G06T 11/26; G06T 2200/24; G06T 2210/12; G06T 2210/41

(57) ABSTRACT

In an illustrative embodiment, systems and methods for visually rendering a representation of periodicity in full resolution physiological data captured by ECG sensors over an extended time period include, for each time period of multiple time periods, representing a respective portion of the full resolution physiological data as a respective series of pixels, and arranging each respective series of pixels in relation to one another to generate a visual image of a time progression of the full resolution physiological data. Arranging each respective series of pixels in relation to one another may include visually depicting a periodicity within the full resolution physiological data as a pixel pattern within the time progression of the full resolution physiological data.

### RELATED APPLICATIONS

This application claims priority to U.S. Provisional Patent Application Ser. No. 63/693,898 entitled “Short Interval Heart Rate Visualization and filed Sep. 12, 2024, which is hereby incorporated by reference in its entirety.

### BACKGROUND

The present disclosure relates to systems, techniques, and computer-readable products featuring visualization techniques for physiological signals indicative of cardiac health, including cardiac arrhythmias.

Heart failure, if left untreated, can lead to certain life-threatening arrhythmias. Both atrial and ventricular arrhythmias are common in patients with heart failure. One of the deadliest cardiac arrhythmias is ventricular fibrillation, which occurs when normal, regular electrical impulses are replaced by irregular and rapid impulses, causing the heart muscle to stop normal contractions. Because the victim has no perceptible warning of the impending fibrillation, death often occurs before the necessary medical assistance can arrive. Other cardiac arrhythmias can include excessively slow heart rates known as bradycardia or excessively fast heart rates known as tachycardia. Cardiac arrest can occur when a patient in which various arrhythmias of the heart, such as ventricular fibrillation, ventricular tachycardia, pulseless electrical activity (PEA), and/or asystole (heart stops all electrical activity), result in the heart providing insufficient levels of blood flow to the brain and other vital organs for the support of life. It is generally useful to monitor heart failure patients to assess heart failure symptoms early and provide interventional therapies as soon as possible.

Patients may be prescribed to wear cardiac treatment devices for extended periods of time.

### SUMMARY OF ILLUSTRATIVE EMBODIMENTS

In one aspect, the present disclosure relates to a system for visually rendering cardiac data, the system including electrocardiogram (ECG) sensors configured to monitor a cardiac status of a subject, and one or more processors configured to perform operations to render a visual representation of periodicity in full resolution physiological data captured by the ECG sensors over an extended period of time including a number of time periods. The operations may include, for each respective time period of the number of time periods, representing a respective portion of the full resolution physiological data of the respective time period as a respective series of pixels, and arranging each respective series of pixels in relation to one another to generate a visual image of a time progression of the full resolution physiological data. Arranging each respective series of pixels in relation to one another may include visually depicting a periodicity within the full resolution physiological data as a pixel pattern within the time progression of the full resolution physiological data.

In some embodiments, the visual representation is a heat map of pixel intensities, where each physiological data value is represented by a respective color. The full resolution physiological data may include electrocardiogram (ECG) sensor signals captured by the ECG sensors. The full resolution physiological data may include cardio-vibrational sensor signals (CVG) captured by cardio-vibrational sensors.

In some embodiments, representing the respective portion of the full resolution physiological data of the respective time period as the respective series of pixels includes converting the respective portion of the full resolution physiological data to a set of values representing differences between topic data points and corresponding time-lagged data points. Each of the topic data points may represent a physiological data value of the full resolution physiological data. Arranging each respective series of pixels in relation to one another may include representing each respective series of pixels of a respective column of a number of side-by-side columns of pixels.

In some embodiments, the periodicity corresponds to at least one of i) one or more cardiac metrics of the subject or ii) one or more cardiac conditions of the subject. The one or more cardiac metrics may include one or more of heart rate, heart rate variability, atrial fibrillation, momentary pauses, or heart rate turbulence. The one or more cardiac conditions may include at least one of supraventricular tachycardia (SVT), ventricular tachycardia, ventricular fibrillation, tachycardia, bradycardia, asystole, a heart pause condition, pulseless electrical activity, atrial fibrillation, or ectopic beat.

In some embodiments, the extended period of time is one of: at least 10 seconds, between 10 seconds and 30 seconds, between 30 seconds and 1 minute, between 1 minute and 5 minutes, between 5 minutes and 10 minutes, between 10 minutes and a half hour, between a half hour and 1 hour, between 1 hour and 2 hours, between 2 hours and 5 hours, between 5 hours and 12 hours, or between 12 hours and 24 hours. The respective time period may be one of: at least one second, between 1 second and 5 seconds, between 5 seconds and 10 seconds, between 10 seconds and 30 seconds, between 30 seconds and 60 seconds, between 1 minute and 5 minutes, between 5 minutes and 10 minutes, between 10 minutes and 20 minutes, or between 20 minutes and 30 minutes. Each pixel of the respective series of pixels now have a first one of a width or a height of at least one pixel and a second one of a width or a height of at least two pixels.

In some embodiments, a wearable cardiac medical device includes the ECG sensors. The wearable cardiac medical device may include a cardiac Holter monitor and associated ECG electrodes. A cardiac monitoring and treatment device may include the ECG sensors. The cardiac monitoring and treatment device may include a wearable cardioverter defibrillator.

In one aspect, the present disclosure relates to a data visualization system for efficiently reviewing cardiac data, the system including physiological sensors configured to monitor a cardiac status of a subject, a non-volatile computer readable medium in operable communication with the physiological sensors, the non-volatile computer readable medium storing full resolution physiological data based on the cardiac or respiration status of the subject, and one or more processors configured to perform operations. The operations may include rendering, at a first reviewer user interface, a visual representation of the full resolution physiological data captured over an extended period of time, where the visual representation includes a pattern indicative of at least one cardiac metric of the subject, receiving, at the first reviewer user interface, a first reviewer input indicative of flagged one or more portions of the physiological data, and rendering, at a second reviewer user interface, a complete representation of the flagged one or more portions of the physiological data.

In some embodiments, the first reviewer user interface includes at least one interactive control for selecting one or more pixel regions within the visual representation of the full resolution physiological data, where the first reviewer input includes at least one set of coordinates corresponding to each pixel region of the one or more pixel regions. The operations may further include, responsive to receiving the first reviewer input, mapping each flagged portion of the flagged one or more portions of the physiological data to a timeframe of the full resolution physiological data. The complete representation of the flagged one or more portions of the physiological data may include at least one graph, each graph of the at least one graph representing a respective timeframe of the full resolution physiological data corresponding to a respective flagged portion of the flagged one or more portions of the physiological data.

In some embodiments, one of the first reviewer interface or the second reviewer interface is provided at a same facility or other location as the subject, and the first reviewer interface is provided in a remote location from the subject. The second reviewer interface may be provided to an authenticated trained clinician or other medical professional.

In some embodiments, the operations further include converting the full resolution physiological data to pixels according to a scale of colors. The operations may further include preparing the visual representation of the full resolution physiological data by, for each respective time period of a number of contiguous time periods within an extended period of time represented in the full resolution physiological data, representing a respective portion of the full resolution physiological data corresponding to the respective time period as a respective series of pixels. The operations may further include arranging each respective series of pixels in parallel to another one of the respective the series of pixels to generate the visual representation of the full resolution physiological data as a parallel arrangement of the series of pixels corresponding to the number of contiguous time periods.

In some embodiments, the operations further include mathematically correlating each respective portion of a number of portions of the full resolution physiological data with a time-lagged copy of the respective portion to produce a number of sets of time-lagged data points. The visual representation of the full resolution physiological data may include a visual mapping of the number of sets of time-lagged data points to corresponding pixels. The physiological sensors may include one or more of a) electrocardiogram (ECG) sensors or b) cardio-vibrational sensors.

In one aspect, the present disclosure relates to a system for visually rendering cardiac physiological data, the system including a non-volatile computer-readable data store configured to retain full resolution physiological data collected by physiological sensors monitoring a subject, and i) hardware logic and/or ii) processing circuitry for executing software code stored to a non-volatile computer-readable medium as instructions. The i) hardware logic and/or ii) the processing circuitry when executing the instructions, may be configured to access the full resolution physiological data spanning an extended period of time, for each respective time period of a number of contiguous time periods within the extended period of time, convert a respective portion of the full resolution physiological data of the respective time period to a respective series of pixels, thereby producing a number of series of pixels, and arrange the number of series of pixels as a visual grid of pixels. Each respective series of pixels may correspond to either a) a respective row of the visual grid in a horizontal grid layout, or b) a respective column of the visual grid in a vertical grid layout. An order of the number of series of pixels within the visual grid may be arranged according to the number of contiguous time periods. The visual grid may include, across at least a portion of the rows of the horizontal grid layout or across at least a portion of the columns of the vertical grid layout, a discernible pattern, where each disruption of the discernible pattern is indicative of one or more cardiac conditions of the subject.

In some embodiments, the arranging is performed in real-time with the collection of the full resolution physiological data. The operations may further include rendering, at a display, the visual grid of pixels for visual identification of any disruption of the discernible pattern. The operations may further include building, in near real-time as new full resolution physiological data is received, an extension of the visual grid including one or more additional rows of the visual grid in the horizontal grid layout, or one or more additional columns of the visual grid in the vertical grid layout, thereby producing a scrolling grid layout of pixels.

In some embodiments, the discernible pattern is indicative of at least one cardiac rhythm. The visual grid may be a heat map of colors selected according to a scale of pixel values. The non-volatile computer-readable data store, the hardware logic and/or the processing circuitry may be cloud-based resources.

In one aspect, the present disclosure relates to a system visually rendering respiratory data, the system including sensors configured to monitor a respiratory status of a subject, and one or more processors configured to perform operations to render a visual representation of periodicity in full resolution physiological data captured by the sensors over an extended period of time including a number of time periods. The operations may include, for each respective time period of the number of time periods, representing a respective portion of the full resolution physiological data of the respective time period as a respective series of pixels. The operations may include arranging each respective series of pixels in relation to one another to generate a visual image of a time progression of the full resolution physiological data, where arranging each respective series of pixels in relation to one another includes visually depicting a periodicity within the full resolution physiological data as a pixel pattern within the time progression of the full resolution physiological data.

In some embodiments, the full resolution physiological data is a collection of pulse oximetry sensor signals, accelerometer signals, spirometer signals, or photoplethysmography (PPG) sensor signals captured by the sensors. The periodicity may correspond to at least one of i) one or more respiratory metrics of the subject or ii) one or more respiratory conditions of the subject. The one or more respiratory metrics may include respiratory rate.

In some embodiments, the one or more respiratory conditions includes sleep apnea. A respiratory monitoring device may include the physiological sensors. The respiratory monitoring device may be one of a nasal clip, an automated ventilator, a ventilation mask, or a tracheal tube.

In one aspect, the present disclosure relates to a cardiac data review system for identifying potential arrhythmias from a wearable medical device, the system including a communication interface configured to receive full-resolution electrocardiogram (ECG) data captured by a wearable medical device monitoring a subject over an extended period of time and computing logic including at least one of i) hardware logic programmed into one or more processing devices or ii) software logic stored to a non-volatile computer readable medium and configured for executing on one or more processors, the computing logic being operably coupled to the communication interface and a display. The computing logic may be configured to segment the full-resolution ECG data into contiguous, fixed-length time periods. The computing logic may be configured to, for each respective time period of the contiguous, fixed-length time periods, perform an autocorrelation on a respective portion of the ECG data corresponding to the respective time period to generate a set of correlation values, and convert the set of correlation values into a corresponding series of pixels, where each pixel has a value determined by a magnitude of a corresponding correlation value. The computing logic may be configured to generate for presentation on the display a visual grid by arranging each series of pixels as a respective column in chronological order, the visual grid configured to present a stable cardiac rhythm as a discernible horizontal pattern and an arrhythmic event as a visual disruption to the discernible horizontal pattern.

In some embodiments, the wearable medical device is a wearable cardioverter defibrillator. The visual grid may be a heat map, and the value of each pixel may be represented by a specific color or intensity from a predefined color scale. The fixed-length time period for segmentation may be between 1 second and 5 seconds. The discernible horizontal pattern may be indicative of a subject's heart rate, and the visual disruption may be indicative of a cardiac condition selected from the group: supraventricular tachycardia (SVT), ventricular tachycardia, ventricular fibrillation, bradycardia, asystole, a heart pause, atrial fibrillation, and an ectopic beat. The computing logic may be configured to receive full-resolution cardio-vibrational sensor (CVG) data and generate a second visual grid based on the CVG data. The system may be embodied in a monitoring station including the display and may be located remotely from the subject.

In one aspect, the present disclosure relates to a data visualization system for efficiently reviewing cardiac data, the system including a communication interface configured to receive, from a wearable cardioverter defibrillator, full-resolution ECG data spanning an extended time period, a display, at least one user input device, and computing logic including at least one of i) hardware logic and/or ii) processing circuitry for executing software code stored to a non-volatile computer-readable medium as instructions, the computing logic being operably coupled to the communication interface, the display, and the at least one user input device. The computing logic may be configured to generate a compact visual representation of the full-resolution ECG data by dividing the ECG data into sequential time intervals, for each time interval, calculating a set of time-lagged correlation values for the ECG data within that time interval, and constructing an image by arranging a series of pixels corresponding to each set of time-lagged correlation values into a sequence of columns. The computing logic may be configured to cause the display to render a first user interface presenting the compact visual representation, the compact visual representation including a visual pattern indicative of a baseline cardiac rhythm. The computing logic may be configured to receive, via the at least one user input device, a user input selecting a region of the compact visual representation showing a disruption in the visual pattern, and, in response to receiving the user input, cause the display to render a second user interface presenting a raw waveform representation of the full-resolution ECG data corresponding to a timeframe of the selected region.

In some embodiments, the computing logic is configured to render the first user interface and the second user interface within a single graphical user interface on the display. The first user interface may be configured for presentation to an ECG technician for triage and the second user interface is configured for presentation to a clinician for diagnosis. The user input may include coordinates of a bounding box drawn around the disruption. The computing logic may be further configured to, prior to constructing the image, apply a high-pass filter to the time-lagged correlation values to accentuate peaks therein. The extended time period may be at least one hour. The compact visual representation may be generated in near real-time as the full-resolution ECG data is received.

In one aspect, the present disclosure relates to computing logic including at least one of i) hardware logic programmed into one or more processing devices or ii) software logic stored to a non-volatile computer readable medium and configured for executing on one or more processors. The computing logic, when executed by a cardiac monitoring system, may cause the cardiac monitoring system to perform operations including accessing full-resolution physiological data captured by a wearable cardiac monitor over an extended period of time, and transforming the full-resolution physiological data into a visual grid, where the transformation includes for each of a collection of sequential time segments of the full-resolution physiological data, calculating a set of autocorrelation values, and arranging pixel representations of each set of autocorrelation values into a series of columns to form the visual grid. The operations may include causing presentation of the visual grid on a display for review, receiving an input identifying a visual disruption of a pattern within the visual grid indicative of a stable physiological rhythm, and in response to receiving the input, initiating a clinical workflow action.

In some embodiments, the clinical workflow action is generating an alert for presentation to a human reviewer. The full-resolution physiological data may include ECG data, the received input may be indicative of ventricular fibrillation, and initiating the clinical workflow action may include sending a command to a wearable cardioverter defibrillator to prepare a therapeutic shock. The operations may further include filtering the set of autocorrelation values according to a filtering parameter to visually accentuate the pattern or the visual disruption in the visual grid. The full-resolution physiological data may further include respiratory data, and the visual disruption may be indicative of a sleep apnea event. Initiating the clinical workflow action may include flagging a portion of the visual grid corresponding to the visual disruption and annotating the flagged portion with a label identifying a suspected cardiac condition.

The foregoing general description of the illustrative implementations and the following detailed description thereof are merely exemplary aspects of the teachings of this disclosure and are not restrictive.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The accompanying drawings, which are incorporated in and constitute a part of the specification, illustrate one or more embodiments and, together with the description, explain these embodiments. The accompanying drawings have not necessarily been drawn to scale. Any values dimensions illustrated in the accompanying graphs and figures are for illustration purposes only and may or may not represent actual or preferred values or dimensions. Where applicable, some or all features may not be illustrated to assist in the description of underlying features. In the drawings:

FIG. 1A through FIG. 1C illustrate block diagrams each representing an example of a different type of full resolution physiological data and its corresponding conversion into an example visual representation of contiguous time periods of the full resolution physiological data;

FIG. 2A and FIG. 2B illustrate a flow chart of an example method for converting full resolution physiological data into a visual representation of contiguous time periods of the full resolution physiological data;

FIG. 3A is a flow diagram of an example process for generating, from raw heart measurements or raw respiration measurements, a visual representation of the full resolution physiological data for review by a user;

FIG. 3B is a flow diagram of an example process for mapping regions of interest selected from the visual representation of the full resolution physiological data to a corresponding portion of the original full resolution physiological data for graphically presenting to a clinician;

FIG. 4A illustrates an example ECG data sample and corresponding visual representation including an example physiological anomaly;

FIG. 4B illustrates another example ECG data sample and corresponding visual representation including an example physiological anomaly;

FIG. 4C illustrates an example heart sounds data sample and corresponding visual representation including an example physiological anomaly;

FIG. 5A and FIG. 5B illustrate a flow chart of an example method for converting individual measurements of full resolution physiological data into pixels for rendering as a visual intensity map;

FIG. 6A through FIG. 6E are block diagrams visually representing example stages of ECG data conversion according to the method of FIG. 5A and FIG. 5B;

FIG. 7 is a block diagram of an example medical device for monitoring a cardiac condition of a patient;

FIGS. 8A-8D illustrate example wearable medical devices for monitoring a cardiac condition of a patient; and

FIGS. 9A-9D are block diagrams visually representing example stages of heart sounds data conversion.

The patent or application file contains at least one drawing executed in color. Copies of this patent or patent application publication with color drawing(s) will be provided by the Office upon request and payment of the necessary fec.

### DETAILED DESCRIPTION OF ILLUSTRATIVE EMBODIMENTS

Medical devices that monitor the cardiopulmonary system obtain a subject's electrocardiogram (ECG) signal from body surface electrodes. Ambulatory wearable defibrillators, such as the Life Vest® Wearable Cardioverter Defibrillator available from ZOLL Medical Corporation of Chelmsford, Mass., use four ECG sensing electrodes in a dual-channel bipolar configuration. This arrangement of ECG sensing electrodes is usually suitable because in most cases it is rare that noise or electrode movement affects the entire body circumference. The dual-channel bipolar configuration provides redundancy and allows the system to operate on a single channel if necessary. Because signal quality also varies from subject to subject, having two channels provides the opportunity to have improved signal pickup, since the ECG sensing electrodes are located in different body positions.

In various implementations, the present disclosure describes systems and methods for the visualization of physiological data. The systems and methods described herein are particularly useful for the efficient review of large volumes of full-resolution data captured by ambulatory medical devices, enabling the rapid identification of physiological events that may require clinical attention.

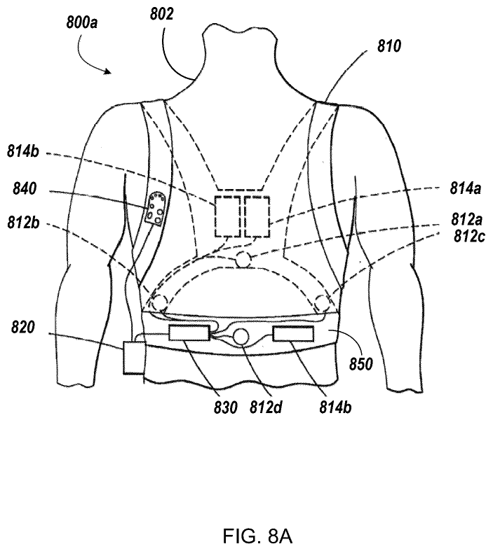

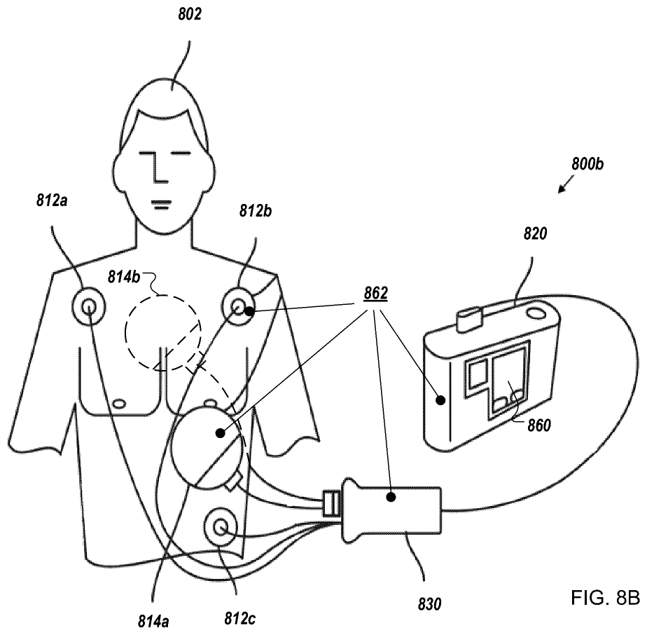

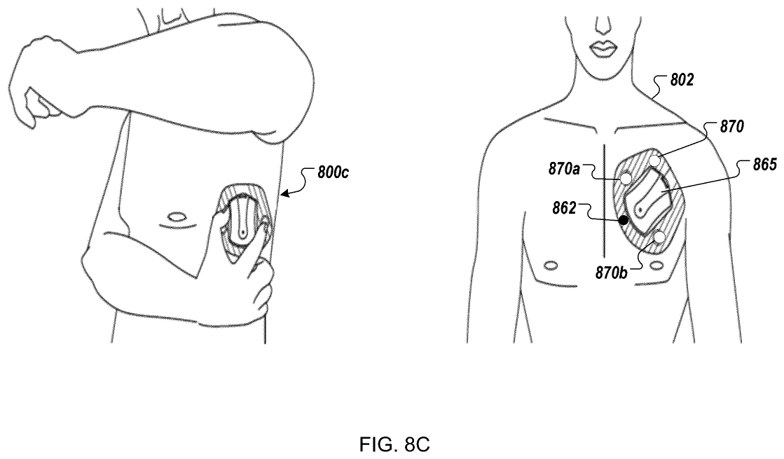

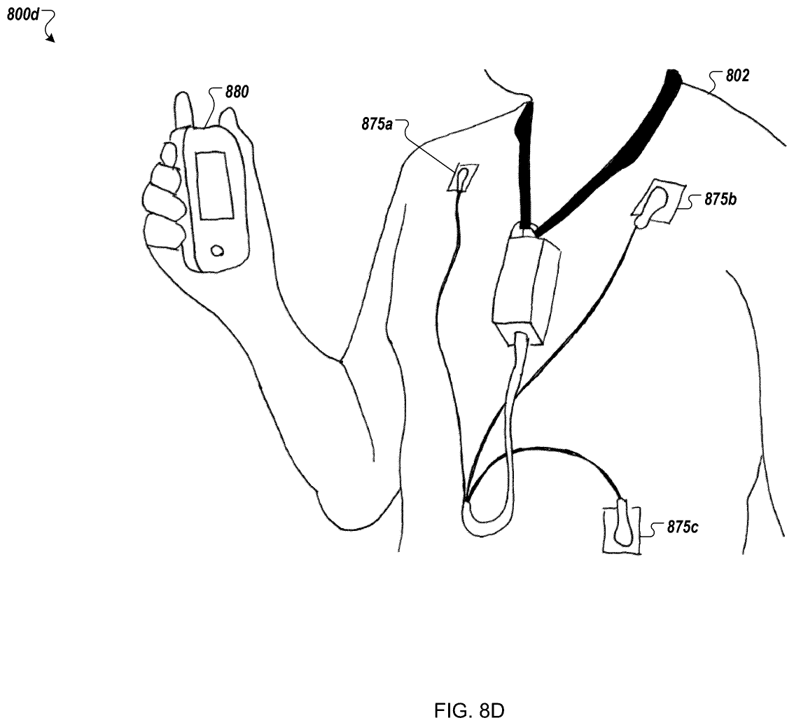

In one implementation, a cardiac data review system is provided for identifying potential arrhythmias from a wearable medical device. The system may be embodied in a monitoring station, which may be physically separate from the patient being monitored. The system includes a communication interface configured to receive full-resolution electrocardiogram (ECG) data captured by a wearable medical device monitoring a subject over an extended period of time. Such a wearable medical device, as illustrated in FIGS. 8A-8D, may be a wearable cardioverter defibrillator. The system includes one or more processors operably coupled to the communication interface and a display. These processors are configured by computer-executable instructions to perform a series of operations as described in further detail below.

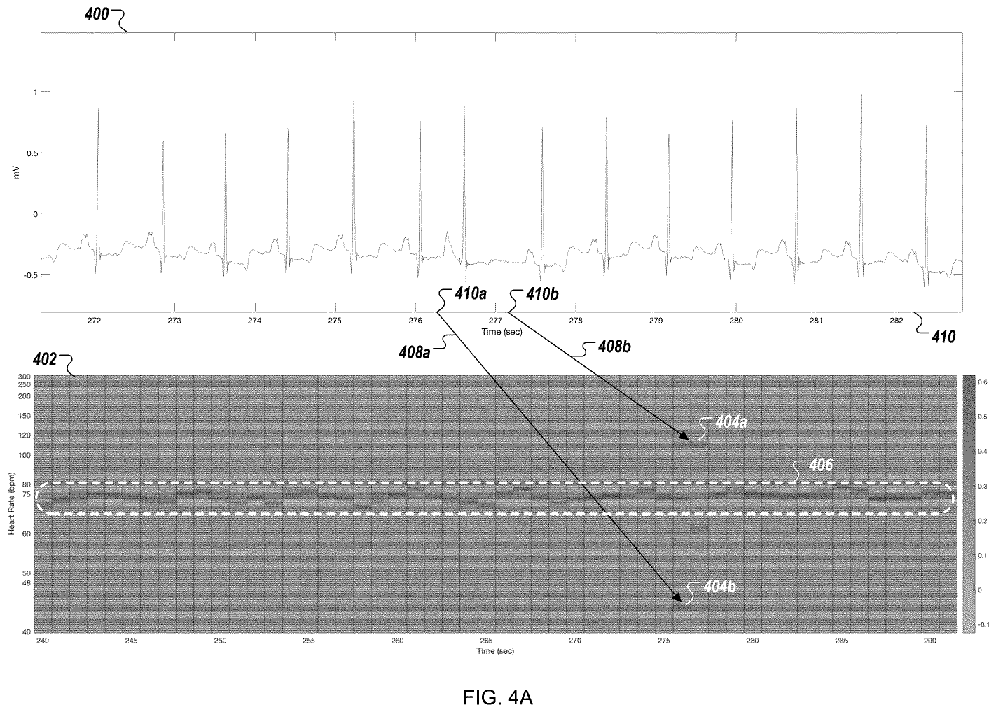

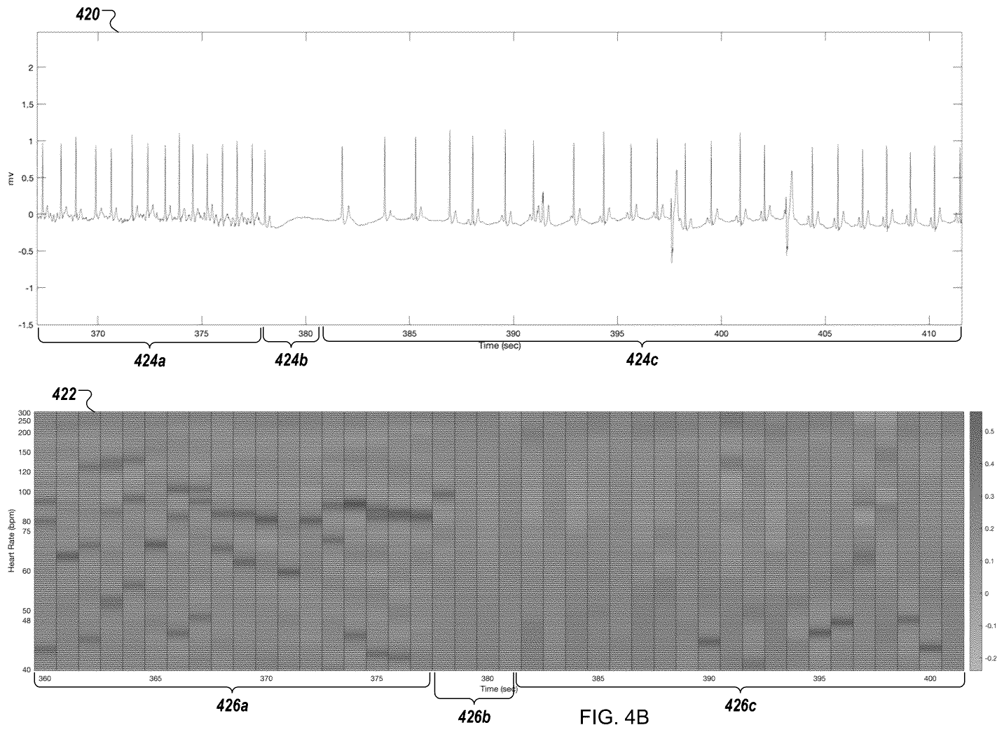

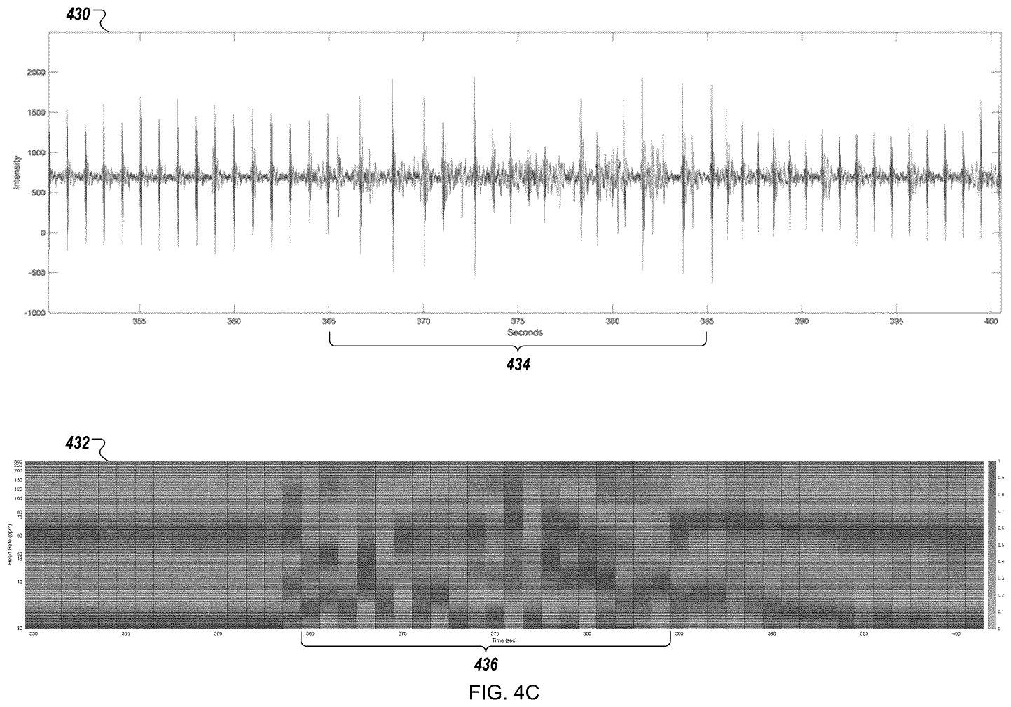

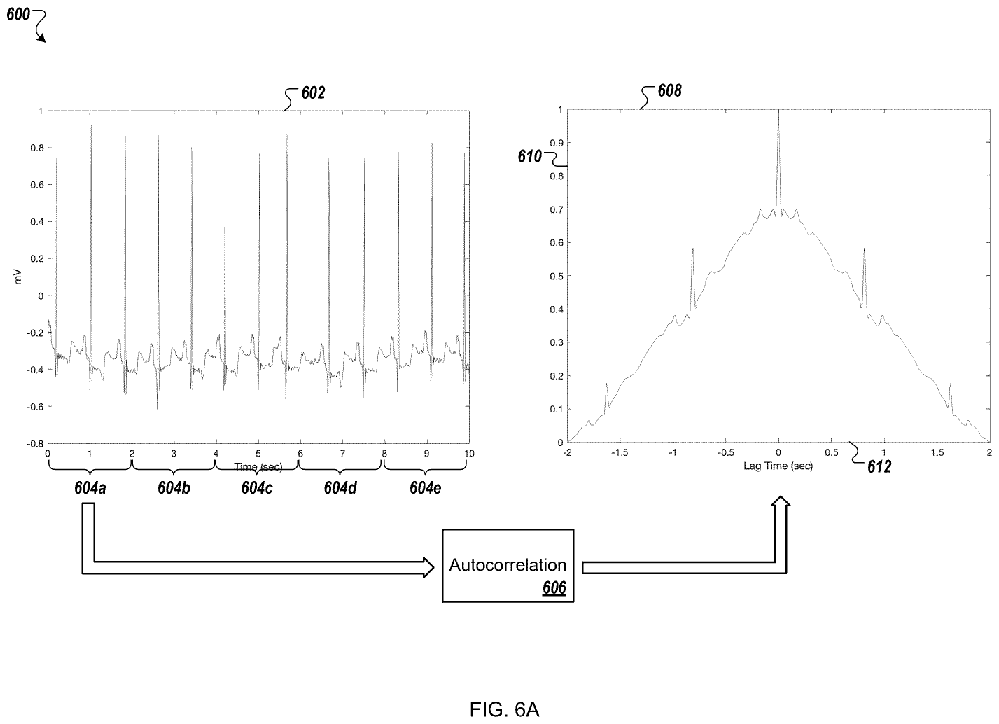

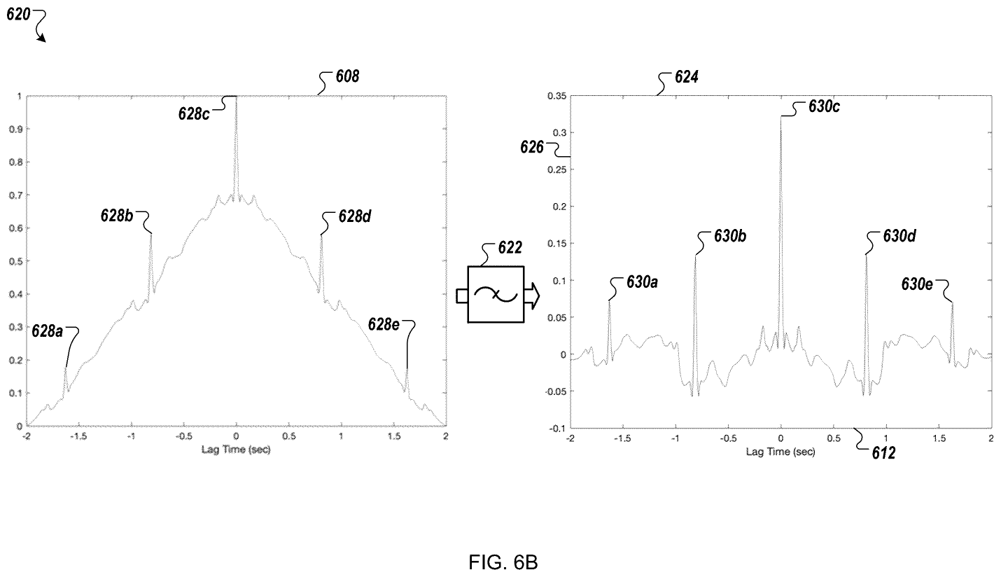

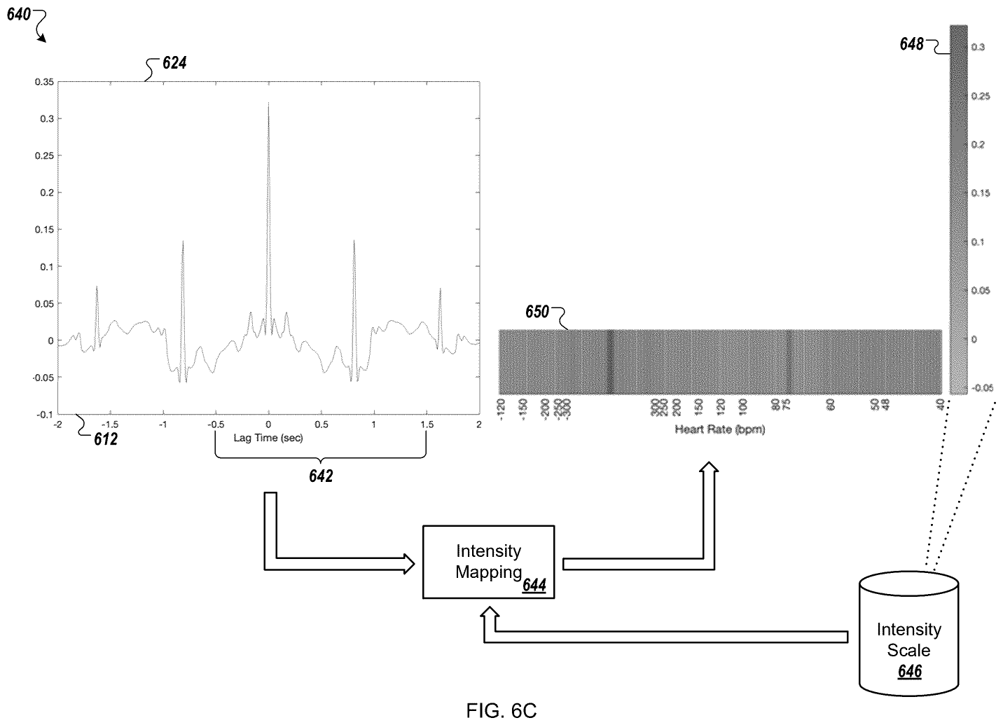

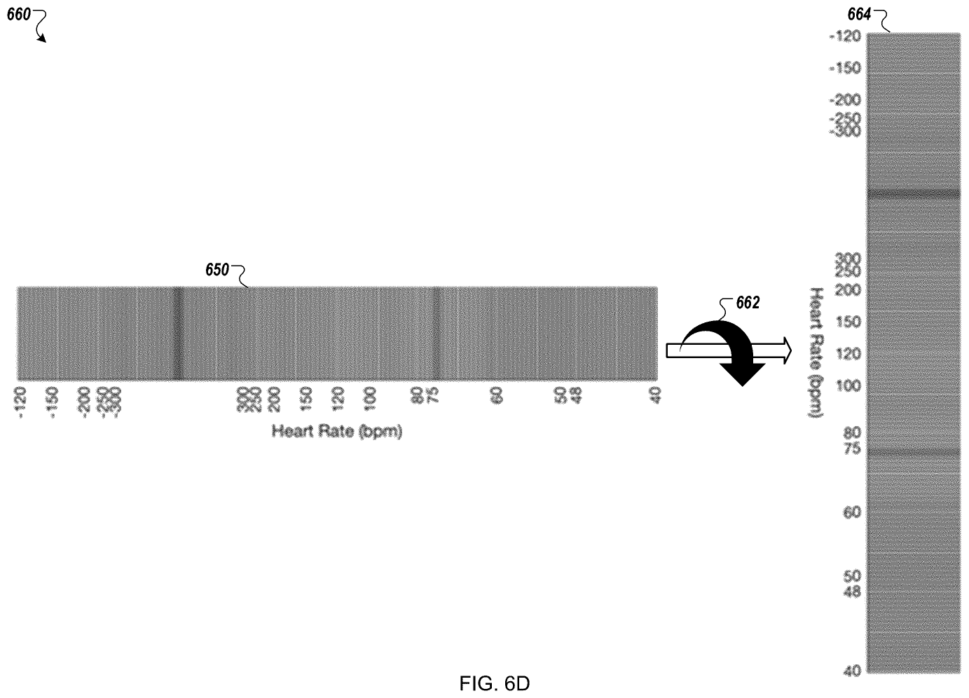

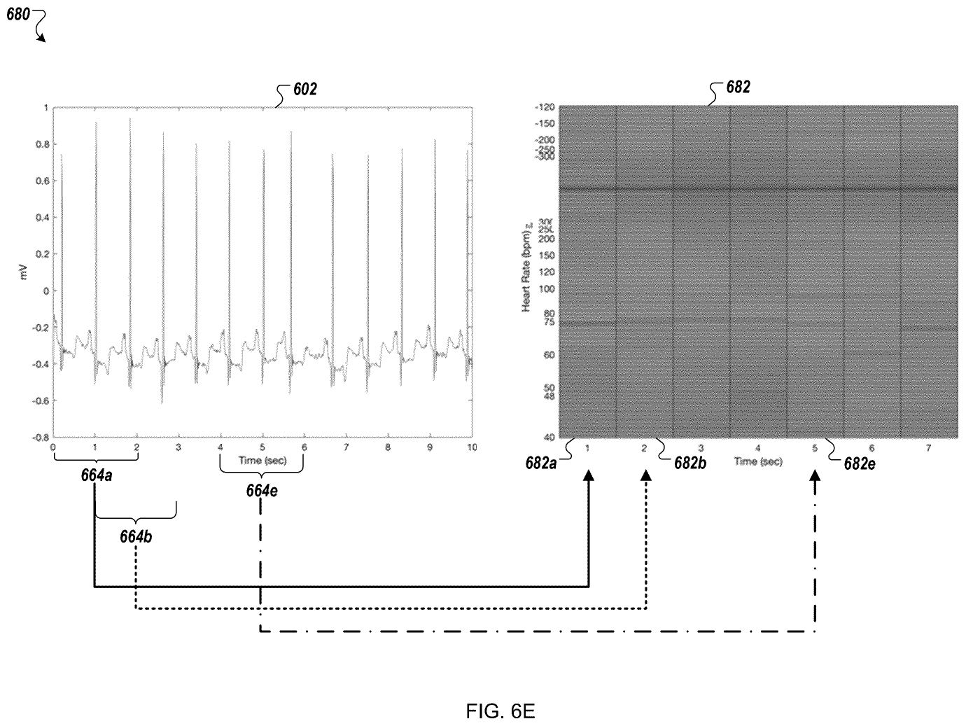

The processor operations include segmenting the full-resolution ECG data into a set of contiguous, fixed-length time periods. As detailed later in this disclosure, this fixed-length period may be between 1 second and 5 seconds, among other ranges. For each respective time period, the processors perform an autocorrelation on a respective portion of the ECG data corresponding to the respective period to generate a set of correlation values. An example autocorrelation process is illustrated in the data conversion example of FIG. 6A and detailed below. The one or more processors can convert the set of correlation values into a corresponding series of pixels, where each pixel has a value determined by a magnitude of a corresponding correlation value. The processors generate for presentation on the display a visual grid by arranging each series of pixels as a respective column, where an order of the collection of series of pixels within the visual grid is arranged according to the set of contiguous time periods. This arrangement, described in further detail below as creating a visual grid of pixels, results in a final image where the visual grid is configured to present a stable cardiac rhythm as a discernible horizontal pattern and an arrhythmic event as a visual disruption to the discernible horizontal pattern. This concept of a discernible pattern and its disruption is visually demonstrated in the examples of FIGS. 4A-4C and further explained below.

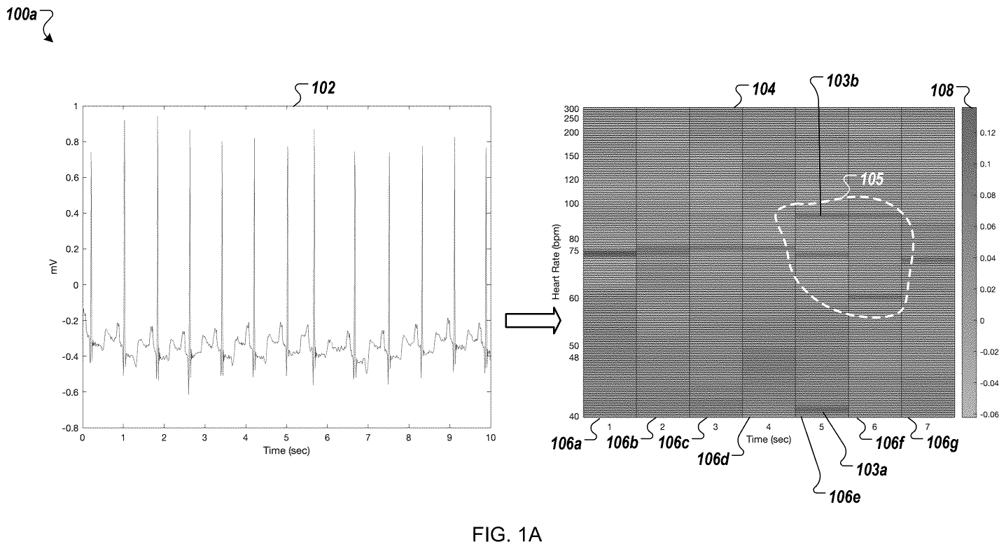

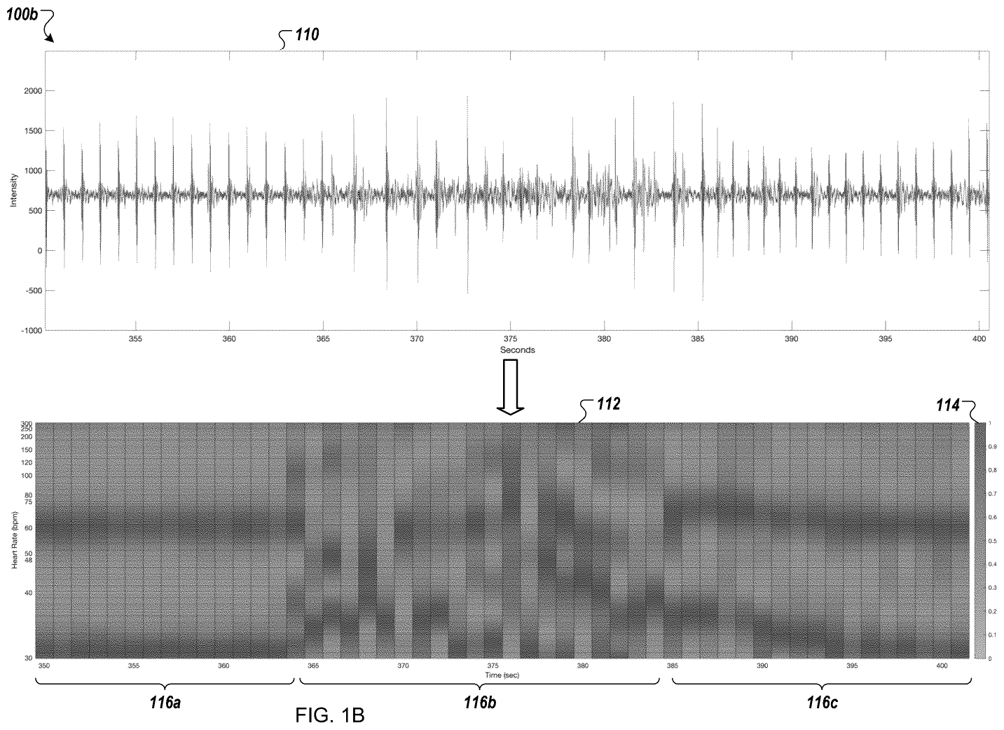

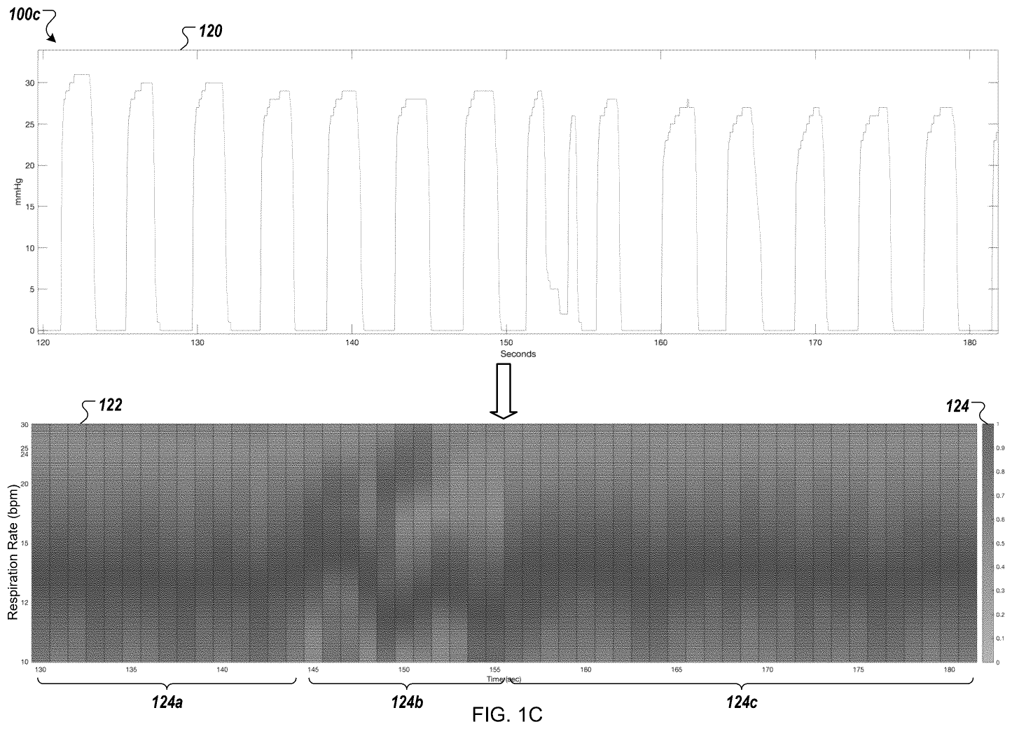

In some implementations, the visual grid is a heat map, and the value of each pixel is represented by a specific color or intensity from a predefined color scale, as supported by the pixel intensity map examples shown in FIGS. 1A-1C. The discernible horizontal pattern may be indicative of a subject's heart rate, and the visual disruption may be indicative of a cardiac condition such as, but not limited to, supraventricular tachycardia (SVT), ventricular tachycardia, ventricular fibrillation, bradycardia, asystole, a heart pause, atrial fibrillation, or an ectopic beat, as enumerated elsewhere in this disclosure. Furthermore, the one or more processors may be further configured to receive and process other data types, for instance, to receive full-resolution cardio-vibrational sensor (CVG) data and generate a second visual grid based on the CVG data, a concept supported by the disclosure's discussion of heart sounds data as shown in the example and associated text for FIG. 1B.

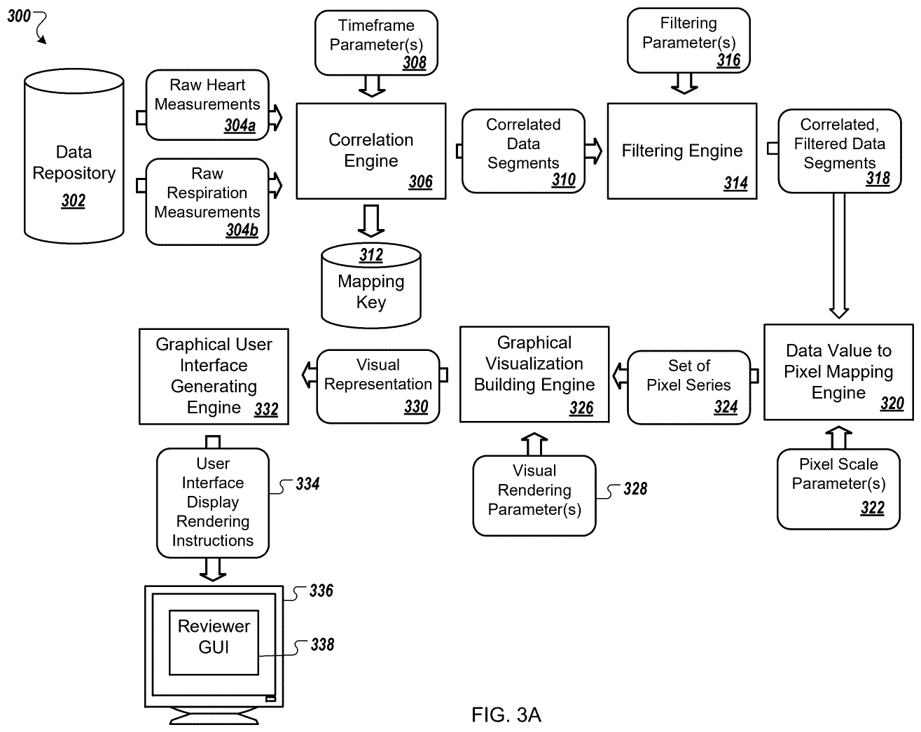

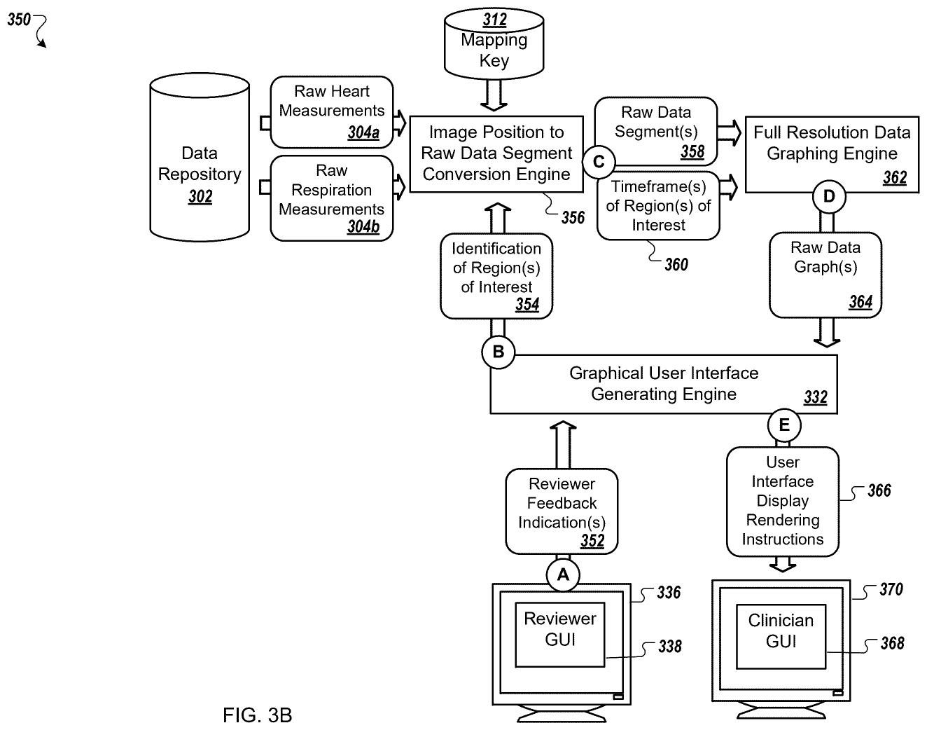

In implementations, a data visualization system is provided for efficiently reviewing cardiac data, which captures the interactive, two-tiered review workflow illustrated in the process flow of FIG. 3B. The system includes a communication interface configured to receive, from a wearable cardioverter defibrillator, full-resolution ECG data spanning an extended time period, a display, at least one user input device, and one or more processors. The processors are configured to execute a series of steps that facilitate this efficient review.

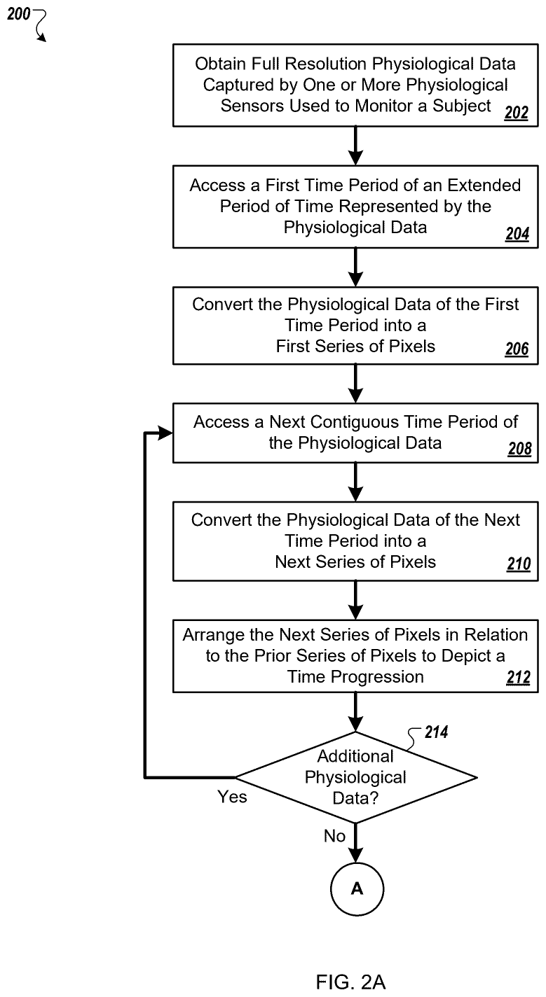

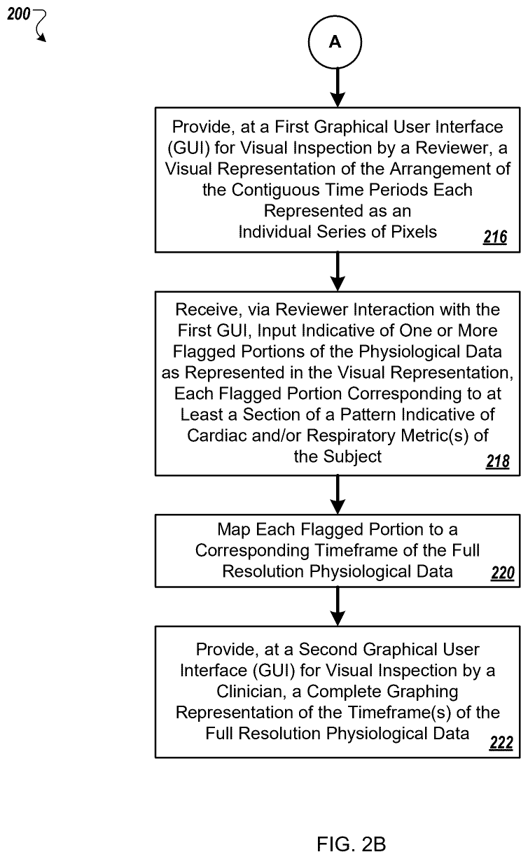

In examples, the processors generate a compact visual representation of the full-resolution ECG data. This generation process includes dividing the ECG data into a set of sequential time intervals, for each time interval, calculating a set of time-lagged correlation values for the ECG data within that time interval, and constructing an image by arranging a series of pixels corresponding to each set of time-lagged correlation values into a sequence of columns. As an example, such a generation process is illustrated in the methods of FIGS. 2A, 3A, and 5A as described in further detail below. In some implementations, the processors are further configured to, prior to constructing the image, apply a high-pass filter to the time-lagged correlation values to accentuate peaks therein. For example, such an operation is illustrated in the data filtering process of FIG. 6B. In some configurations, the generation of this compact visual representation may be performed in near real-time as new data is received.

In further operations, the processors cause the display to render a first user interface presenting the compact visual representation. Such compact visual representation includes a visual pattern indicative of a baseline cardiac rhythm. The system is configured to receive, via the at least one user input device, a user input selecting a region of the compact visual representation showing a disruption in the visual pattern. This interactive selection process allows, within a clinical workflow, a reviewer to provide input indicative of flagged one or more portions of the data as illustrated in further detail below. As such, the user input may, for example, include coordinates of a bounding box drawn around the disruption, an interaction consistent with selecting a region of interest.

In further operations, in response to receiving the user input, the processors cause the display to render a second user interface presenting a raw waveform representation of the full-resolution ECG data corresponding to a timeframe of the selected region. This two-stage display process, involving a first reviewer user interface and a second reviewer user interface illustrates the disclosed clinical workflow. The system may be configured such that the first user interface and the second user interface are rendered within a single graphical user interface on the display. Further, the system may be configured such that the first user interface is configured for presentation to an ECG technician for triage and the second user interface is configured for presentation to a clinician for diagnosis. This two-tiered user model is referenced in more detail below with reference to a layperson or other medical assistant performing an initial review to flag data for a trained clinician or other medical professional.

In yet some examples, the concepts of this disclosure are embodied as a non-transitory computer-readable medium storing instructions that, when executed, cause a cardiac monitoring system to perform a method for enabling clinical action. The operations include accessing full-resolution physiological data captured by a wearable cardiac monitor over an extended period of time. The operations then transform the full-resolution physiological data into a visual grid, where the transformation includes, for each of a set of sequential time segments, calculating a set of autocorrelation values and arranging pixel representations of each set into a series of columns to form the visual grid. The instructions may further cause the system to filter the set of autocorrelation values according to a filtering parameter to visually accentuate the pattern or the visual disruption in the visual grid, as performed by the filtering engine shown in FIG. 3A and described in detail below.

The operations further include causing presentation of the visual grid on a display for review and receiving an input identifying a visual disruption of a pattern within the visual grid indicative of a stable physiological rhythm. In response to receiving the input, the operations initiate a clinical workflow action. This workflow action may include generating an alert for presentation to a human reviewer, a function for flagging data for attention as described in further detail below. In another example, the action may include flagging a portion of the visual grid corresponding to the visual disruption and annotating the flagged portion with a label identifying a suspected cardiac condition, which is the functional outcome of clinician interpretation.

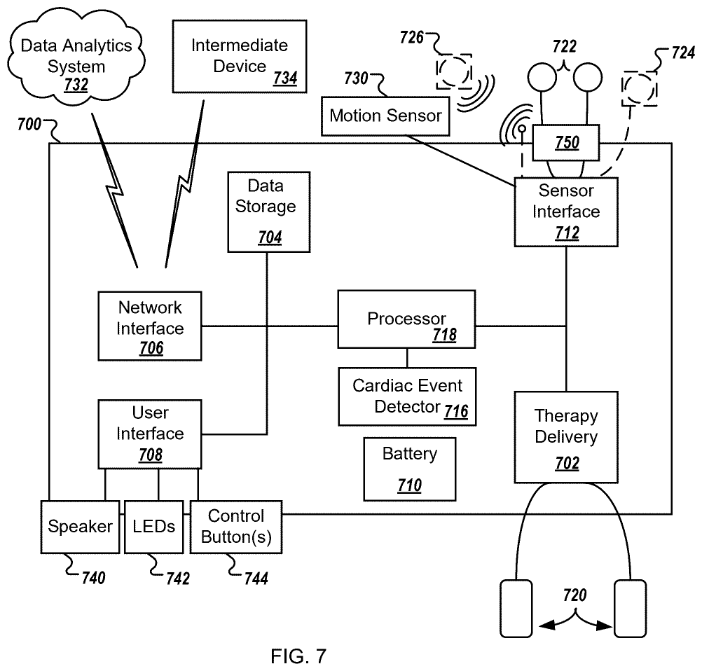

In some applications, where the physiological data includes ECG data and the received input is indicative of ventricular fibrillation, initiating the clinical workflow action may include sending a command to a wearable cardioverter defibrillator to prepare a therapeutic shock. Such applications connect the identification of a life-threatening arrhythmia as described in this disclosure with the therapy delivery capabilities of the device as shown in the system diagram of FIG. 7. Further, where the primary data described herein is cardiac data, the methods are also applicable to other data types. For instance, the physiological data may further include respiratory data, and the visual disruption may be indicative of a sleep apnea event, as discussed and illustrated in the respiration data example of FIG. 1C.

Heart rhythms may also be monitored using vibrational sensors (e.g., including acoustic sensors and/or audio transducers) to detect and record cardio-vibrational signals and the timing of the cardio vibrations, including any one or all of S1, S2, S3, and S4 cardio vibrations. Other cardio-vibrational parameters which may be monitored by recording cardio-vibrational signals include electromechanical activation time (EMAT), percentage of EMAT (% EMAT), systolic dysfunction index (SDI), and left ventricular systolic time (LVST). EMAT is generally measured from the onset of the Q wave on the ECG to the closure of the mitral valve within the S1 cardio vibration. Prolonged EMAT has been associated with reduced left ventricular ejection fraction (LVEF, being a measure of how much blood is being pumped out of the left ventricle of the heart with each contraction). % EMAT is EMAT corrected for heart rate. % EMAT is related to the efficiency of the pump function of the heart. SDI is a multiplicative combination of ECG and sound parameters (EMAT, S3, QRS duration, and QR interval). SDI predicts left ventricular systolic dysfunction with high specificity. LVST is defined as the time interval between the S1 and the S2 cardio vibrations. It is the systolic portion of the cardiac cycle. LVST has some heart rate dependence and tends to be approximately 40% (range 30-50%) of the cardiac cycle but is affected by disease that produces poor contractility and/or a low ejection fraction.

The description set forth below in connection with the appended drawings is intended to be a description of various, illustrative embodiments of the disclosed subject matter. Specific features and functionalities are described in connection with each illustrative embodiment; however, it will be apparent to those skilled in the art that the disclosed embodiments may be practiced without each of those specific features and functionalities.

Reference throughout the specification to “one embodiment” or “an embodiment” means that a particular feature, structure, or characteristic described in connection with an embodiment is included in at least one embodiment of the subject matter disclosed. Thus, the appearance of the phrases “in one embodiment” or “in an embodiment” in various places throughout the specification is not necessarily referring to the same embodiment. Further, the particular features, structures or characteristics may be combined in any suitable manner in one or more embodiments. Further, it is intended that embodiments of the disclosed subject matter cover modifications and variations thereof.

It must be noted that, as used in the specification and the appended claims, the singular forms “a,” “an,” and “the” include plural referents unless the context expressly dictates otherwise. That is, unless expressly specified otherwise, as used herein the words “a,” “an,” “the,” and the like carry the meaning of “one or more.” Additionally, it is to be understood that terms such as “left,” “right,” “top,” “bottom,” “front,” “rear,” “side,” “height,” “length,” “width,” “upper,” “lower,” “interior,” “exterior,” “inner,” “outer,” and the like that may be used herein merely describe points of reference and do not necessarily limit embodiments of the present disclosure to any particular orientation or configuration. Furthermore, terms such as “first,” “second,” “third,” etc., merely identify one of a number of portions, components, steps, operations, functions, and/or points of reference as disclosed herein, and likewise do not necessarily limit embodiments of the present disclosure to any particular configuration or orientation.

Furthermore, the terms “approximately,” “about,” “proximate,” “minor variation,” and similar terms generally refer to ranges that include the identified value within a margin of 20%, 10% or preferably 5% in certain embodiments, and any values therebetween.

All of the functionalities described in connection with one embodiment are intended to be applicable to the additional embodiments described below except where expressly stated or where the feature or function is incompatible with the additional embodiments. For example, where a given feature or function is expressly described in connection with one embodiment but not expressly mentioned in connection with an alternative embodiment, it should be understood that the inventors intend that that feature or function may be deployed, utilized or implemented in connection with the alternative embodiment unless the feature or function is incompatible with the alternative embodiment.

In one aspect, the present disclosure relates to a unique graphical format for visualizing full resolution cardiac data in an arrangement that compresses the information into a compact graphical format of pixel values (e.g., intensities, hues, etc.). The visual rendering of cardiac data in the compact graphical format may highlight patterns in the data in a manner that is discernable without the intensive training and experience required to analyze, for example, a common electrocardiogram (ECG) data graph to recognize similar metrics, rhythms, and/or anomalies. For example, rather than requiring review by a clinician to identify a portion of data that may be of interest, the compact graphical format may be reviewed by a relative layperson who can then flag the identified pattern for interpretation by a clinician, thereby freeing up clinician time and/or supporting faster identification of potential problems.

In one aspect, the present disclosure relates to a graphical data format capable of drawing patterns out of full resolution physiological data without the need to first analyze the physiological data to identify an underlying rhythm in the data, such as heartbeat detection. The visualization may be prepared using full resolution physiological data divided into a set of periodic time intervals of an arbitrary (and relatively small) length of time. The underlying rhythm may be expressed in the graphical data format as a relatively unbroken line or other pattern of similar pixel values (e.g., intensity, hue, etc.) spanning a majority of the periodic time intervals presented in the graphical data format. Disruptions in the underlying rhythm, for example, may be quickly visually discernable as a break or disruption in the otherwise generally regular periodic pattern. In this manner, the graphical data format may avoid distortions in the visualization that would otherwise be caused by errors in detection of the underlying rhythm (e.g., beat detection).

The periodic patterns, in some examples, may each correspond to a separate cardiac metric, cardiac condition, respiratory metric, and/or respiratory condition in a subject. The periodic patterns may be indicative of at least one cardiac rhythm. The cardiac metrics may include, in some examples, heart rate, heart rate variability, atrial fibrillation, momentary pauses, and/or heart rate turbulence. Respiratory metrics may include respiration rate. Monitoring of the periodicity of the patterns within the graphical data format, for example, may allow a layperson or clinician to swiftly recognize artifacts that could be indicative of a problem in the subject's cardiac status (e.g., one or more cardiac conditions) such as, in some examples, supraventricular tachycardia (SVT), ventricular tachycardia, ventricular fibrillation, tachycardia, bradycardia, asystole, a heart pause condition, pulseless electrical activity, atrial fibrillation, and/or ectopic beat (e.g., premature ventricular contraction (PVC)). In another example, monitoring of the periodicity of the patterns within the graphical data format may allow a layperson or clinician to swiftly recognize artifacts that could be indicative of a problem in the subject's respiratory status (e.g., respiration status, one or more respiratory conditions, etc.) such as sleep apnea. The reviewer who recognized the artifact(s), for example, could then flag the section of the data for further review (e.g., by a clinician or medical professional). The further review may include reviewing the original full resolution physiological data corresponding to a timeframe of the identified artifact(s).

FIG. 1A through FIG. 1C illustrate example physiological data sets and corresponding conversions to graphical data format. Although the example physiological data of FIG. 1A through FIG. 1C is represented over a limited span of time, this is primarily for visual clarity in the format of the present disclosure and should not be mistaken to represent a preferred embodiment. A benefit of the graphical data format is its ability to highlight anomalies within the physiological measurements even when a lengthy period of time is compressed into a graphic format that may be reproducible in the space of a computer monitor or medical device display screen. In practice, the physiological data represented in the graphical data format may include an extended period of time of minutes, tens of minutes, up to an hour, multiple hours, or even an entire day.

Turning to FIG. 1A, in a first physiological data conversion example 100a, data measurements of a sample of ECG data 102, captured over the span of 10 seconds, have been converted into a graphical data format 104 representing a time series of data values divided into periodic time intervals 106a-g of one second each. The data of each periodic time interval 106a-g has been coded as pixel values according to a pixel intensity map 108.

As can be speedily recognized through visual interpretation, a bright pink band spans approximately 75 bpm through seconds one through five and seven of the graphical data format 104 (e.g., time intervals 106a-e and 106g). However, in the sixth time interval 106f, two separate bright pink bands stand out against the generally blue background of pixel values, one band at approximately 98 bpm and another band at approximately 61 bpm, while the pixel values at and surrounding 75 bpm are coded in shades of blue. A layperson or other medical assistant, for example, may be relied upon after minimal training to recognize this disruption in the overall heartbeat pattern and flag the discrepancy for the attention of a clinician. Trained medical personnel, such as a clinician, may recognize this discrepancy as a premature beat at five seconds (e.g., pink in a faster heart rate region), followed by a compensatory pause at six seconds (e.g., pink in a lower heard rate region).

Turning to FIG. 1B, in a second physiological data conversion example 100b, data measurements of example heart sounds data 110, captured over the span of fifty seconds (e.g., from second 350 to second 400), have been converted into a graphical data format 112 representing a time series of data values divided into periodic time intervals of one second each. The data of each periodic time interval has been coded as pixel values according to a pixel intensity map 114.

As is easily discerned through visual interpretation, a set of generally parallel and horizontal bright pink bands span approximately 30 bpm and 60 bpm through a first timespan 116a of seconds 350 to 362 of the graphical data format 112. In a second timespan 116b from second 364 to second 384, however, the bright pink color is scattered among a generally blue background. A layperson or other medical assistant, for example, may be relied upon after minimal training to recognize a disruption in the overall sounds pattern represented in the second timeframe 116b and flag the discrepancy for the attention of a clinician. Further, turning to a third timespan from second 385 to second 401, while the set of two bright pink bands have reappeared, the color differentiation is not as clear as during the first time period 116a and the bands are sloping rather than generally horizontal. When faced with this visual pattern, again, a layperson or other medical assistant may be relied upon after minimal training to recognize a disruption in the overall sounds pattern represented in the third timeframe 116c and flag the discrepancy for the attention of a clinician.

Turning to FIG. 1C, in a third physiological data conversion example 100c, data measurements of example respiration data 120, captured over the span of sixty seconds (e.g., from second 120 to second 180), have been converted into a graphical data format 122 representing a time series of data values divided into periodic time intervals of one second each. The data of each periodic time interval has been coded as pixel values according to a pixel intensity map 124.

As is visibly evident, a wide and somewhat irregular but generally horizontal bright pink band spans approximately 12 bpm and 17 bpm through a first timespan 124a of seconds 130 through 144 of the graphical data format 122 and a third timespan 124c of seconds 156 through 180. However, in a second timespan 124b from second 145 to second 155, the bright pink color spikes up into the 23-25 pbm region and creates a “C”-shaped artifact breaking up the otherwise generally horizontal band. A layperson or other medical assistant, for example, may be relied upon after minimal training to recognize a disruption in the overall respiration pattern represented in the second timeframe 124b and flag the discrepancy for the attention of a clinician.

FIG. 2A and FIG. 2B illustrate a flow chart of an example method 200 for converting full resolution physiological data into a visual image representation of contiguous time periods of the full resolution physiological data. The method 200, for example, may be performed to produce the graphical format 104 of the ECG data sample 102 of FIG. 1A, the graphical format 112 of the heart sounds data sample 110 of FIG. 1B, and/or the graphical format 122 of the respiration data sample 122 of FIG. 1C.

In some implementations, the method 200 begins with obtaining full resolution physiological data captured by one or more physiological sensors used to monitor a subject (202). In one example, the full resolution physiological data contains ECG signals captured by one or more ECG electrodes (ECG sensors). The ECG signals (e.g., measurements), for example, may be obtained from skin-facing ECG electrodes integrated into or connected to a wearable cardiac monitoring device, a cardiac Holter monitor, or a cardiac monitoring and treatment device (e.g., with an automated external defibrillator or a wearable cardioverter defibrillator). The signals may be obtained by one or more processors of the wearable medical device. The signals, in another example, may be relayed via a network to a remote computing system (e.g., a system in operable communication with the sensor(s)), such as a medical facility server or a set of cloud-based resources of a cloud computing platform where portions of the method 200 are performed. The full resolution physiological data, in one example, contains data measurements captured through a cardio-vibrational sensor monitoring a patient's heart (e.g., cardio-vibrational signals (CVG) or cardio-vibrational sensor signals). The cardio-vibrational sensor, in some examples, may be a medical grade accelerometer or microphone configured to monitor pulmonary vibrations. The vibrational sensor, in one example, may be attached to or built into a wearable cardiac monitoring device. The vibrational sensor may be positioned to contact skin on the patient being monitored. In another example, the vibrational sensor is releasably attached to the patient, for example using a medical grade adhesive. The cardio-vibrational signals may be obtained from the vibrational sensor, for example, by one or more processors of a medical device, such as a wearable cardiac monitoring device. The cardio-vibrational signals, in another example, may be relayed from the vibrational sensor via a network to a remote computing system such as a medical facility server or a cloud computing platform where portions of the method 200 are performed. In another example, the full resolution physiological data may represent respiration vibrations. The physiological data may be captured, for example, by a respiratory monitoring device, such as, in some examples, a nasal clip, an automated ventilator, a ventilation mask, and/or a tracheal tube, including one or more sensors for capturing respiration vibrations. The one or more sensors, for example, may include one or more pulse oximetry sensors, one or more accelerometers (e.g., accelerometer 862 of the heart failure management system (HFMS) device 800c of FIG. 8C), one or more spirometers, and/or one or more photoplethysmography (PPG) sensors. The physiological data, for example, may be a collection of pulse oximetry sensor signals, accelerometer signals, spirometer signals, or PPG sensor signals. The pulse oximetry sensor signals, accelerometer signals, spirometer signals, and/or PPG sensor signals may be obtained from the corresponding sensor(s), for example, by one or more processors of a respiratory monitoring device. The pulse oximetry sensor signals and/or PPG sensor signals, in another example, may be relayed from the corresponding sensor(s) via a network to a remote computing system such as a medical facility server or a cloud computing platform where portions of the method 200 are performed.

In some implementations, a first time period of an extended period of time represented by the physiological data is accessed (204). In some embodiments, the full resolution physiological data is captured locally, for example by one or more sensors in wired or wireless connection to a medical device performing at least portions of the method 200. accessing the physiological data, for example, may involve receiving, from the one or more sensors, physiological measurements in real-time or near-real-time to their capture. In some embodiments, the full resolution physiological data is transferred from a medical device or other computing device, via a network, from a point of capture of the physiological measurements. The network transfer may occur in real-time or after some delay. For example, the physiological measurements may be retained in a non-volatile computer-readable data store and batch transferred or streamed in real-time/near real-time to a network server or cloud computing environment from a medical setting or wearable device used during a patient's typical daily activities. Accessing the first time period, in these embodiments, may involve accessing the physiological measurements from a storage region of a non-volatile computer readable medium (e.g., storage device).

The first time period, in some examples, may be at least one second, between one second and five seconds, between five seconds and ten seconds, between ten seconds and thirty seconds, between thirty seconds and sixty seconds, between one minute and five minutes, between five minutes and ten minutes, between ten minutes and twenty minutes, or between twenty minutes and thirty minutes. A length of the first time period, for example, may be determined in part upon a total length of the physiological data such that the physiological data may be evenly divided by the length of the first time period. For example, in the event of batch transfer of data, the physiological data may be represented as a series of images, each image representing the data obtained in a single batch. In another example, the data may be stored data captured during an earlier timeframe. In this manner, a total length of the data may be known. The extended period of time, for example, may be at least ten seconds, between ten seconds and thirty seconds, between thirty seconds and one minute, between one minute and five minutes, between five minutes and ten minutes, between ten minutes and a half hour, between a half hour and one hour, between one hour and two hours, between two hours and five hours, between five hours and twelve hours, or between twelve hours and an entire day (e.g., twenty-four hours) of data measurements. The length of the first time period, in another example, may be based in part on a maximum number of contiguous time periods to allow the entire data set to be represented within a single viewing screen. In a further example, the length of the first time period may be set to a default value such that pixel patterns in the data render in a consistent manner, thereby allowing for easily recognizable interruptions in the pixel pattern that a clinician or layperson can be trained to rapidly evaluate. As illustrated in each of the examples 100a, 100b, and 100c of FIG. 1A through FIG. 1C, for example, the length of the first time period is one second.

In some implementations, the physiological data of the first time period is converted into a first series of pixels (206). As illustrated in relation to FIG. 1A through FIG. 1C, for example, individual physiological measurements may be converted to pixel color values (e.g., individual tones and/or hues) according to a heat map of pixel intensities, such as the heat map 108 of FIG. 1A, the heat map 114 of FIG. 1B, or the heat map 124 of FIG. 1C. In additional examples, the pixels take on particular values among a set of tints of one or more colors, a set of tones of one or more colors, and/or a set of color shades. In further embodiments, the pixel values may each be represented by a particular shading or fill. For example, although described as a single pixel such as a single dot, each physiological data measurement may be represented as a pixel block including two or more pixels, where the pixel block has a fill value (e.g., color, shading pattern, fill pattern, etc.). For example, as illustrated in each of FIG. 1A through FIG. 1C, the individual pixels in the series of pixels may be rendered to the screen as rectangular blocks (pixel blocks or pixel regions) each assigned a different pixel value. Although described in relation to the physiological measurements, in some embodiments, the measurements of the physiological data may be prepared (e.g., filtered, screened, or otherwise adjusted) prior to conversion to pixel values, as discussed in greater detail below.

In some implementations, a next contiguous time period of the physiological data is accessed (208), and the physiological data of the next contiguous time period is converted into a next series of pixels (210). The physiological data of the next contiguous time period, for example, may be converted in the same manner as described in relation to the first time period at operation 206. Converting each physiological data measurement of the first time period to a corresponding pixel of the first series of pixels, for example, may include storing the first series of pixels to a non-volatile computer-readable medium as an array of pixel values. In another example, the pixels may be stored to a separate image or as an additional portion of an existing image as a series of pixel regions or pixel blocks.

In some implementations, the next series of pixels is arranged in relation to the prior series of pixels to depict a time progression (212). The next series of pixels, for example, may be arranged in parallel to the prior series of pixels (e.g., horizontally or vertically) to produce a parallel arrangement of series of pixels. The next series of pixels may abut the prior series of pixels, or a gap may be placed between series of pixels. As illustrated in the graphical data format 104 of FIG. 1A, the graphical data format 112 of FIG. 1B, and the graphical data format 122 of FIG. 1C, for example, each series of pixels may be visually represented as pixel blocks arranged as side-by-side columns of contiguous physiological data representations.

The accessing (208), converting (210), and arranging (212), in some implementations, repeats for the remainder of the physiological data (214).

Turning to FIG. 2B, in some implementations, a visual representation of the arrangement of the contiguous time periods each represented as an individual series of pixels is provided at a first graphical user interface (GUI) for inspection by a reviewer (216). The visual representation presented at the first reviewer user interface, in some embodiments, is a static representation (e.g., having a defined beginning and end). The static representation, for example, may represent a discrete data set. In some embodiments, the visual representation is a dynamic representation, including new data being added as it is obtained and processed. For example, the dynamic representation may be a scrolling representation or provide the opportunity to shift to a different timeframe (e.g., using a scroll bar). In this manner, a clinician, assistant, or other personnel may actively monitor the visual representation for signs of an anomaly. The visual representation may be presented in the vicinity of the subject (e.g., on a medical device display or a screen connected to one or more medical devices monitoring the subject). For example, medical facility staff or other caretakers (e.g., staff at an assisted living facility, rehabilitative facility, or convalescent hospital) may monitor the visual representation when in a same room as the subject. In another example, the visual representation may be presented at a remote computing device disposed outside of the immediate region of the subject. In illustration, the remote computing device may be positioned at a main desk of a hospital floor or a separate building or facility from the subject's location.

In some implementations, an input indicative of one or more flagged portions of the physiological data as represented in the visual representation is received via reviewer interaction with the first GUI (218). Each flagged portion, for example, may correspond to at least a section of a pattern indicative of cardiac and/or respiratory metrics of the subject (218). The first GUI, for example, may be interactive such that a user may select a section of the visual representation, such as by clicking on or touching a portion of the visual representation. The input indicative of the one or more flagged portions, in some examples, may include a set of coordinates corresponding to each pixel region or block of at least one pixel region or block, a time or time range, and/or or an identifier of each pixel of one or more pixels selected.

Turning to FIG. 1A, in an illustrative example, a reviewer may interact with the graphical data format 104 to select at least a portion of the information presented in column 106e and/or column 106f. Selecting the section of the visual representation, for example, may include causing at least one data point, such as a first anomalous pixel 103a or a second anomalous pixel 103b of the column 106e, to be identified. To assist in selection, in some embodiments, the reviewer is provided the ability to zoom into a portion of the screen to more accurately highlight a portion of the information. The user may be provided the ability to bulk select (e.g., “color over” or highlight portions of the pixels of the visual representation) or draw a border around a section of the data, such as a section 105 of FIG. 1A. In another example, the user may be provided the ability to position a dual-sided scroll bar to the section of the timeline of interest (e.g., from second 5 to second 6). In an additional example, the user may be provided at least one interactive control, such as a time range control or a text box, to enter a range of times corresponding to the timeframe of the data presented within the visual representation.

Returning to FIG. 2B, in some implementations, each flagged portion is mapped to a corresponding timeframe of the full resolution physiological data (220). The mapping, for example, may include translating each reviewer input to a time or time range of the underlying full resolution physiological data.

In some implementations, a complete graphing representation of the corresponding timeframe(s) of the full resolution physiological data is provided at a second GUI for visual inspection by a clinician (222). The clinician, for example, may be authenticated and/or trained in analyzing the full resolution physiological data. In some examples, the clinician may be a doctor, surgeon, or other medical professional. The second GUI may be presented at a same computing device or a different computing device. The computing devices used for presenting the first GUI and the second GUI may be remotely located from each other (e.g., in a different room, a different building, or at a large geographic distance such as miles apart). The complete representation, in some illustrative examples, may include at least a portion of the sample of ECG data 102 of FIG. 1A, the sample heart sounds data 110 of FIG. 1B, and/or the sample respiration data 120 of FIG. 1C. Each corresponding timeframe may be presented in a same graph or in a separate graph. If multiple timeframes have been identified, the physiological data values between each set of timeframes may or may not be rendered. For example, a threshold portion of leading and/or following data may be presented in relation to each timeframe.

Although described in relation to the corresponding timeframes, in some embodiments, the full resolution physiological data may include a larger portion of the physiological data used to generate the visual representation of the first GUI. When presenting a greater amount of the full physiological data, in some embodiments, the corresponding timeframe(s) of the flagged portions may be highlighted within the second GUI. In some examples, the flagged portion(s) may be represented with a different background color, graphed with a different line color, encircled, enlarged, and/or visually highlighted along an x-axis (e.g., a different color and/or rendering of the numeric identifiers of the time range of each timeframe, a colored bar along the time range of each timeframe, etc.).

In some embodiments, the second GUI is interactive such that the clinician may scroll along the graph of the full resolution physiological data and/or zoom into portions of the full resolution physiological data. The interaction, in another example, may allow the clinician to pan back and forth between each flagged timeframe.

Although described in relation to a particular set of operations, in other embodiments, the method 200 includes more or fewer operations. For example, when reviewer interactions (218) represent one or more timeframes, the mapping (220) may be omitted. Additionally, although the operations of the method 200 are present in a particular order, in other embodiments, certain operations may be performed in a different order and/or concurrently. For example, prior to arranging the series of pixels (e.g., in a graphical format) (212), each time period may be converted (206, 208) into a separate series of pixels (e.g., in a vector format). The time periods, in another example, may be divided out prior to conversion, and portions of the physiological data converted concurrently (206, 208). Other modifications of the method 200 are possible.

FIG. 3A is a flow diagram of an example process 300 for generating, from raw heart measurements or raw respiration measurements, a visual representation of the full resolution physiological data for review by a user. The process 300, for example, may perform at least portions of the method 200 of FIG. 2A. The process 300, for example, illustrates various components of a data visualization system for efficiently reviewing cardiac data. The various engines of the process 300, in some embodiments, are configured as software routines or algorithms (e.g., at least a portion of a software program) coded as instructions for executing on processing circuitry, such as one or more processors. Certain engines or operations performed by certain engines, in some embodiments, are configured as hardware logic hard-coded or programmed into processing circuitry, such as a programmable logic chip.

In some implementations, the process 300 begins with accessing raw physiological data measurements 304 (e.g., raw heart measurements 304a and/or raw respiration measurements 304b) from a data repository 302. The raw physiological data measurements 304, for example, may represent data values as captured by one or more sensors monitoring physiological patterns of a patient.

In some implementations, a correlation engine 306 accesses one or more timeframe parameters 308 for dividing the raw physiological data 304 into a set of data segments. The timeframe parameters 308, for example, may include a segment length (e.g., a timeframe), a maximum total number of segments, and/or a maximum number of physiological data values per segment. The timeframe parameters 308 may include one or more user-customizable parameters. For example, a user may select, for example via portal settings, medical device settings, and/or a user interface page in which the graphical format will be displayed) a segment length to define the coarseness or fineness of presentation of the raw physiological data 304 in graphical format. In another example, the timeframe parameters 308 may include one or more parameters set based at least in part on a target display for the graphical data format. For example, parameters may differ for a relatively small medical device display in comparison to a display of a portable computing device (e.g., tablet or laptop computer) and/or a larger stationary computer monitor positioned at a desk. The correlation engine 306 may divide the raw physiological measurements 304 according to the rules set forth by the timeframe parameter(s) 308.

In some embodiments, to enable rapid conversion from a graphical data format to the raw physiological measurements, the correlation engine 306 stores a mapping key 312 between the raw physiological data measurements 304 and each of the divided data segments (e.g., a starting measurement, a range of measurements within the stored raw physiological data 304, etc.). The mapping key 312, for example, may be applied at a later time in mapping between a graphical format of the raw physiological data 304 and the original raw physiological data 304.

The correlation engine 306, in some embodiments, mathematically correlates each respective data segment of the divided data segments with a time-lagged copy of the respective data segment (e.g., time-lagged data points) to produce a set of correlated data segments 310 having values which represent differences between the topic data points of the segmented raw physiological measurement data values and corresponding time-lagged data points. For example, the correlation engine 306 may mathematically superimpose, upon each respective data segment, increasing fractions of the respective data segment to calculate a respective similarity measurement between each of the raw physiological data values of the respective data segment and each data value of the superimposed lagged time frame of physiological data values overlapping with at least a portion of the respective data segment.

In some implementations, a filtering engine 314 filters the correlated data segments 310 according to one or more filtering parameters 316. The filtering parameters 316, in some examples, may be designed to accentuate ranges of physiological data values outside of the typical/normal/anticipated range(s), thereby resulting in more pronounced patterns of periodicity in the resultant graphical format. For example, a high-pass filter may be applied according to a floor threshold filtering parameter (e.g., lowest passing value) to flatten the data range, thereby focusing on main peaks within the physiological data values. In other examples, a low-pass filter may be applied according to a ceiling threshold filtering parameter to accentuate negative peaks, or a stop-band filter may be applied according to both a floor threshold value and a ceiling threshold value to accentuate both low and high peaks.

In some embodiments, the filtering parameters 316 include band pass filtering parameters to remove outlier data values (e.g., data values which are within an unreasonable range based on the expected range corresponding to the raw physiological data measurements 304). Outliers (e.g., data values that make no sense beyond a problem with the sensor), for example, may be set to a default value designed to avoid the data point being color-coded as part of a periodic pattern since it is recognized as untrustworthy data. Conversely, the raw physiological data 304 may be filtered prior to correlation by the correlation engine 306 to replace untrustworthy data points with default values.

The filtering parameters 316, in some embodiments, include historic metrics and/or values of the patient such that a corresponding filter applied to the physiological data will result in drawing out differences between a baseline of the patient and current data values. For example, a floor threshold value and/or a ceiling threshold value may be set according to one or more historic physiological metrics for the patient such as, in some examples, mean or average physiological data values, historic high (e.g., peak) physiological data values, and/or historic low (e.g., valley) physiological data values. The filtering parameters 316, in some examples, may be set within a threshold number of one or more historic physiological metric values, within a percentile difference from one or more historic physiological metric values, and/or at one or more historic physiological metric values.

In some implementations, the filtering engine 314 produces, by filtering the correlated data segments 310 according to the filtering parameter(s) 316, a set of correlated, filtered data segments 318. The data values of the correlated, filtered data segments 318, for example, may continue to have a one-to-one correspondence to the raw physiological measurements 304. Rather than removing any data points, for example, the filtering engine 314 may simply adjust the range(s) of the data values within the correlated data segments 310 with the goal of accentuating periodic patterns within the graphical format of the raw physiological data 304.

The data values within the correlated filtered data segments 318, in some implementations, are mapped to pixel values by a data value to pixel mapping engine 320. The data value to pixel mapping engine 320, for example, may map each data value of each data point of each data segment within the correlated filtered data segments 318 to a corresponding pixel value according to one or more pixel scale parameters 322, thereby producing a visual mapping of the data points of each data segment to corresponding pixels. The pixel scale parameters 322, for example, may define or influence correspondence between data point values and pixel values. For example, the pixel scale parameters 322 may be used to define the pixel intensity map 108 of FIG. 1A, the pixel intensity map 114 of FIG. 1B, and/or the pixel intensity map 124 of FIG. 1C. A portion of the pixel scale parameters 322 may be user customized. The pixel scale parameters 322 may identify, from a set of options, a preferred pixel scale (e.g., range and/or set of tones and/or hues). A user, for example, may be provided the option to select from a set of pixel scale options, similar to having the ability to select a color scheme in a software platform or application. The pixel scale parameters 322 may set a total number of different potential pixel values as an absolute value (e.g., fifty) or a relative value (e.g., a different pixel value per each set of twenty contiguous potential data point values). The pixel scale parameters 322 are influenced, in some embodiments, by the filtering parameter(s) 316. For example, all values at or within a set range of one of the filtering threshold parameters may be set to a particular first color of a pixel heat map (e.g., including, for example, a range of hues or tones of the first color), such as the blue tones illustrated in the heat map 108 of FIG. 1A), while all values at or within a set range of a maximum bound (e.g., bandpass filtered or otherwise a maximum anticipated range of potential data values) may be set to a particular second color of the pixel heat map (e.g., including, for example, a range of hues or tones of the second color), such as the pink tones illustrated in the heat map 108 of FIG. 1A.

In some implementations, according to a pixel scale defined or influenced by the pixel scale parameter(s) 322, the data value to pixel mapping engine 320 generates a set of pixel series 324, each series corresponding to a given segment of the correlated, filtered data segments 318. The set of pixel series 324, in some examples, may be arranged as a set of vectors or a grid.

In some implementations, a graphical visualization building engine 326 generates a graphical format of the set of pixel series 324 in accordance with one or more visual rendering parameters 328. The one or more visual rendering parameters 328, in some examples, may include a width and/or height to apply in rendering each pixel value in the set of pixel series 324 (e.g., as a rectangle, square, etc. of the color/hue/shade of the pixel value), a sizing of the visual rendering of the graphical format (e.g., a screen footprint, a full-screen indication, a scrolling window format, etc.), a spacing (e.g., between each pixel series 324 and/or between each rendered pixel), and/or a formatting (e.g., png, jpeg, svg, etc.). The width may be at least one pixel, at least two pixels, etc., while the height may be at least one pixel, at least two pixels, etc. A portion of the visual rendering parameters 328 may be customizable by an end user, such as a width of each column of the graphical data format 104 of FIG. 1A. The user, for example, may be provided with settings and/or an interactive user interface for inputting visual rendering parameters 328. The graphical visualization building engine 326, for example, may create a visual grid of pixel values according to the set of pixel series 324 and the visual rendering parameter(s) 328, where each pixel series of the set of pixel series 324 is rendered as a column or row of the visual grid. For example, the graphical visualization building engine 326 may produce a horizontal grid layout and/or a vertical grid layout, depending on the visual rendering parameters 328. The graphical visualization building engine 326 may provide a visual representation 330 of the set of pixel series 324 to a graphical user interface generating engine 332.

In some implementations, the graphical user interface generating engine 332 generates user interface display rendering instructions 334 for presenting the visual representation 330 in a reviewer graphical user interface 338 on a display 336 of a computing device. The user interface display rendering instructions 334, in some examples, may include instructions for rendering in a browser, portal application, or software tool executing on the computing device having the display 336.

Turning to FIG. 3B, a flow diagram illustrates an example process 350 for mapping regions of interest selected, via the reviewer GUI 338, from the visual representation 330 of the full resolution physiological data to a corresponding portion of the original full resolution physiological data 304 for graphically presenting to a clinician. The process 350, for example, may execute portions of the method 200 of FIG. 2B. The various engines of the process 350, in some embodiments, are configured as software routines or algorithms (e.g., at least a portion of a software program) coded as instructions for executing on processing circuitry, such as one or more processors. Certain engines of the process 350 or operations performed by certain engines, in some embodiments, are configured as hardware logic hard-coded or programmed into processing circuitry, such as a programmable logic chip.

In some implementations, the process 350 begins with a reviewer submitting, via the reviewer GUI 338, one or more reviewer feedback indications 352. The feedback indications, for example, may be submitted in one of the manners described in relation to operation 218 of the method 200 of FIG. 2B.

In some implementations, the graphical user interface generating engine 332 provides the identification of one or more regions of interest 354 for use by an image position to raw data segment conversion engine 356. The indications, for example, may be similar in form to options described in relation to operation 218 of the method 200 of FIG. 2B.

In some implementations, the image position to raw data segment conversion engine 356 converts the received identification of region(s) of interest 354 to timeframes of the raw physiological data measurements 304. The image position to raw data segment conversion engine 356, for example, may use the mapping key 312 produced by the correlation engine 306 of FIG. 3A to translate between the identification of region(s) of interest 354 and one or more time portions of the raw physiological data measurements 304.

Upon identifying the one or more timeframes of the regions of interest 360, in some embodiments, the image position to raw data segment conversion engine 356 collects one or more raw data segments 358 of the raw physiological data measurements 304 from the data repository 302 corresponding to the one or more time portions. The raw data segment(s) 358 may include additional data measurements, such as a time buffer (e.g., absolute or percentage) surrounding each region of interest 354. If two or more regions of interest 354 are sufficiently close along the timeline (e.g., to be presented together in a single screen as a raw data graph), in some implementations, the image position to raw data segment conversion engine 356 collects the raw physiological data measurements 304 within a timeframe between each set of regions of interest 354 of the two or more regions of interest 354. In other implementations, for example where the entire set of raw physiological data measurements 304a may be reasonably reviewed within a single screen and/or represents up to a threshold time period that could be comfortably reviewed by a clinician without losing sight of the region(s) of interest 354, the image position to raw data segment conversion engine 356 collects the raw physiological data measurements 304 in its entirety.

In some implementations, the image position to raw data segment conversion engine 356 provides the raw data segment(s) 358 and the timeframe(s) of the region(s) of interest 360 for use by a full resolution data graphing engine 362 to produce at least one raw data graph 364 representing the original raw physiological data measurements 304. The raw data graph(s) 364, for example, may appear similar to the sample of ECG data 102 of FIG. 1A, the heart sounds data sample 110 of FIG. 1B, or the sample respiration data 120 of FIG. 1C, depending upon the type of data.

In some implementations, the raw data graph(s) 364 are provided for use by the graphical user interface generating engine 332 for preparing user interface display rendering instructions 366 for use in displaying the raw data graph(s) 364 in a clinician graphical user interface 368 at a display 370. The user interface display rendering instructions 366, in some examples, may include instructions for rendering in a browser, portal application, or software tool executing on the computing device having the display 370. Although illustrated as different GUI presentations 338, 368 presented at different displays 336, 370, in some embodiments, the same reviewer may submit the reviewer feedback indication(s) 352 for use in generating the clinician GUI 368 on the display 336.

In illustration, the process 300 of FIG. 3A and the process 350 of FIG. 3B may be used to focus in on anomalies discovered when reviewing a grid-formatted version of raw physiological data, as shown in FIG. 4A through FIG. 4C. Turning to FIG. 4A, an example raw physiological data graph 400 of an ECG data sample is illustrated along with a corresponding visual grid 402 generated using the raw physiological data measurements. The visual grid 402 includes example disruptions 404a, 404b in a discernible pattern 406. A set of arrows 408a, 408b lead from approximately a start point 410a and an end point 410b along a timeline 410 of the physiological data graph 400 to the corresponding disruptions 404a, 404b produced by the anomaly in the ECG pattern exhibited between the start point 410a and the end point 410b. The disruptions 404a, 404b, for example, may be indicative of a cardiac condition of the subject (e.g., premature ventricular contraction (PVC)).

Turning to FIG. 4B, an example raw physiological data graph 420 of another ECG data sample is illustrated along with a corresponding visual grid 422 generated using the raw physiological data measurements. Three time portions 424a, 424b, and 424c of the raw physiological data measurements depicted in the data graph 420 are illustrated along the timeline of the data graph 420. In the first time portion 424a, the data may represent a state of atrial fibrillation (e.g., abnormal heartbeat). In a corresponding time portion 426a of the visual grid 422, a scattered pattern of pink blocks among the blue visually represents the atrial fibrillation cardiac condition. In the second time portion 424b of the data graph 420, a pause in heart activity occurs, represented in a corresponding time portion 424b of the visual grid 422 as a single pink block followed by generally blue. Finally, in a third time portion 424c, a nodal rhythm is presented in the data graph 420. The nodal rhythm is represented, in a corresponding time portion 426c of the visual grid 422, as a period of time with some pink but with no discernable pattern. Since throughout the heart sounds data sample represented by the example raw physiological data graph 420, no normal (e.g., healthy) pattern is present, no discernable pattern is recognizable within the visual grid 422.

Turning to FIG. 4C, an example raw physiological data graph 430 of a heart sounds data sample is illustrated along with a corresponding visual grid 432 generated using the raw physiological data measurements. A time portion 434 is marked on the timeline of the raw physiological data graph 430 indicating a period of time of atrial fibrillation. In a corresponding time portion 436 of the visual grid 432, two generally horizontal pink bands spanning a remainder of the data graph 430 are disrupted (e.g., from about 365 seconds to about 385 seconds). During the time portion 436, the visual grid 432 lacks a consistent visual pattern.

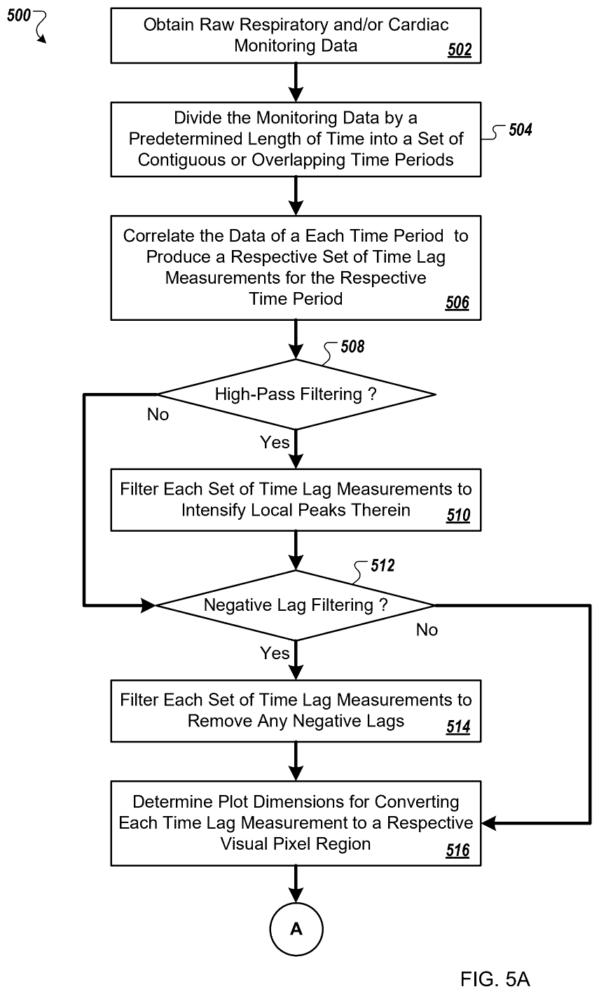

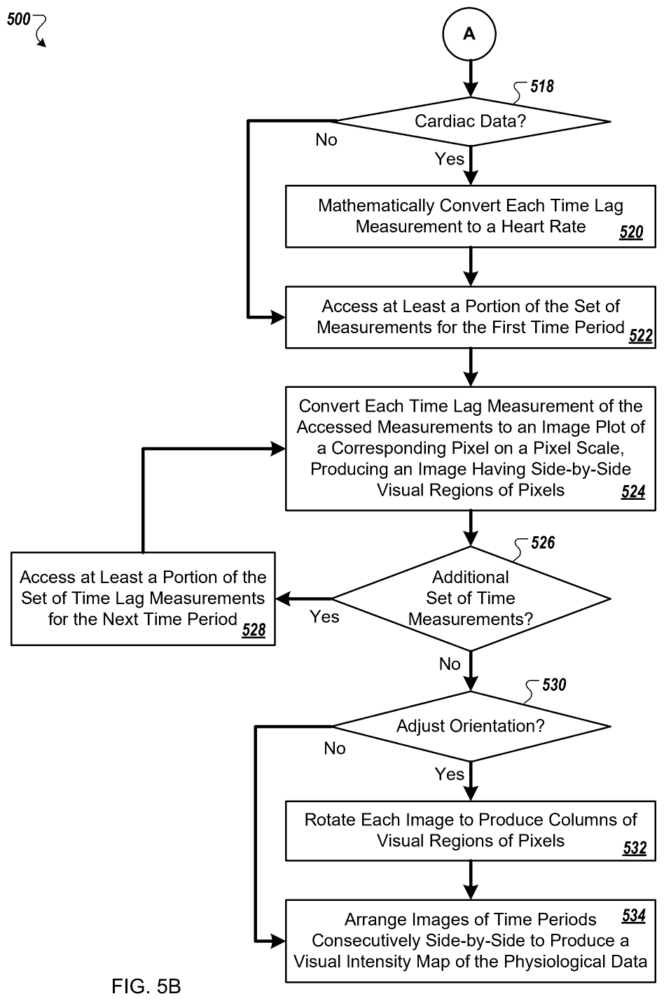

FIG. 5A and FIG. 5B present a flow chart of an example method 500 for converting individual measurements of full resolution physiological data into pixels for rendering as a visual intensity map as illustrated in step-by-step block diagrams of stages of data conversion illustrated in FIG. 6A through FIG. 6D. The method 500, for example, provides a particular, detailed example of a portion of the more general process 300 discussed in relation to FIG. 3A.

In some implementations, the method 500 begins with obtaining raw respiratory and/or cardiac monitoring data (502). The raw physiological data, in some examples, may be obtained as described in relation to the operation 202 of the method 200 of FIG. 2A and/or the raw heart measurements 304a and the raw respiration measurements 304b of FIG. 3A.

In some implementations, the monitoring data is divided by a predetermined length of time into a set of contiguous or overlapping time periods (504). The data may be divided, for example, as described in relation to the correlation engine 306 of FIG. 3A. In some embodiments, portions of data are duplicated between time periods. For example, the time periods may be divided into segments including a first segment corresponding to seconds 0-2 of a data timeline, a second segment corresponding to seconds 1-3 of the data timeline, a third segment corresponding to seconds 2-4 of the date timeline, and so on.

In some implementations, the data of each time period is correlated to produce a respective set of time lag measurements for the respective time period (506). The data may be correlated, for example, as described in relation to the correlation engine 306 of FIG. 3A.

Turning to FIG. 6A, a block diagram illustrates an example data conversion 600 involving applying an autocorrelation operation 606 to each time period 604a-e of a set of contiguous time periods 604 (e.g., two second increments of data measurements) of cardiac monitoring data 602. The autocorrelation operation 606, for example, may access the first time period 604a of the cardiac monitoring data 602 (e.g., from second zero to second two) and calculate, using a sliding time lag window of the first time period 604a over the same data set of the first time period 604a, a degree of similarity between the data set of the first time period 604a and its time-lagged version. The autocorrelation operation 606, for example, produces a data set of autocorrelated data measurements 608 (e.g., a set of time lag measurements) having a same number of individual data points as the data segment of the first time period 604a. As illustrated, the autocorrelated data measurements 608 present a degree of similarity 610 (e.g., 1 being identical, when there is no time lag between the first time period 604a and its time-lagged copy) over lag time 612 (e.g., −2 seconds to 2 seconds). The same autocorrelation operation 606 may be repeated for the remaining time periods 604b-e of the cardiac monitoring data 602.

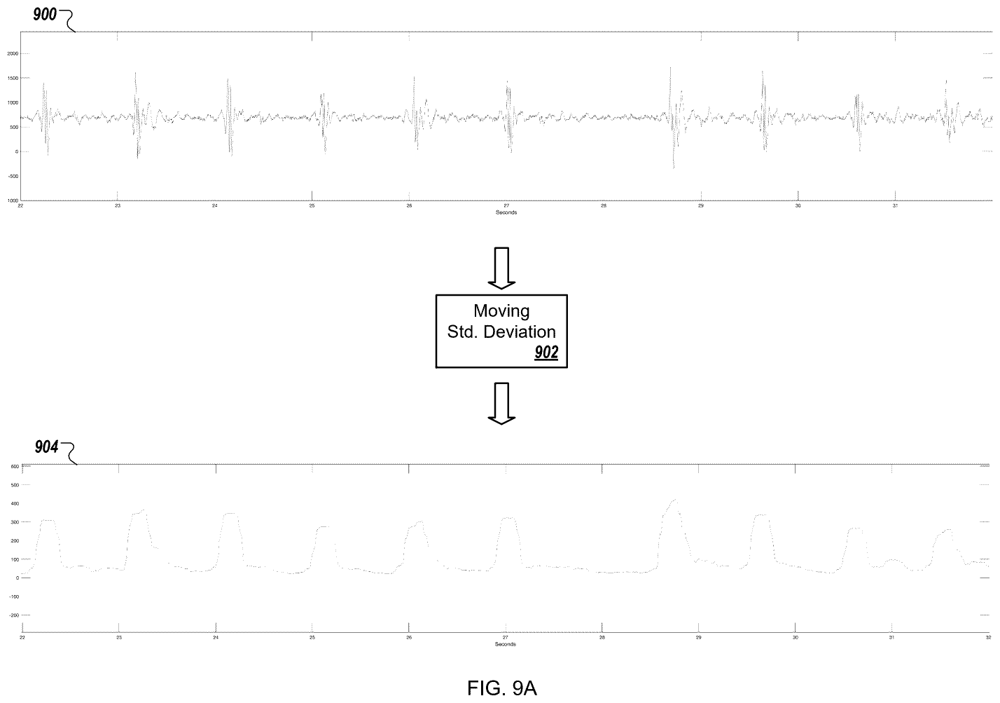

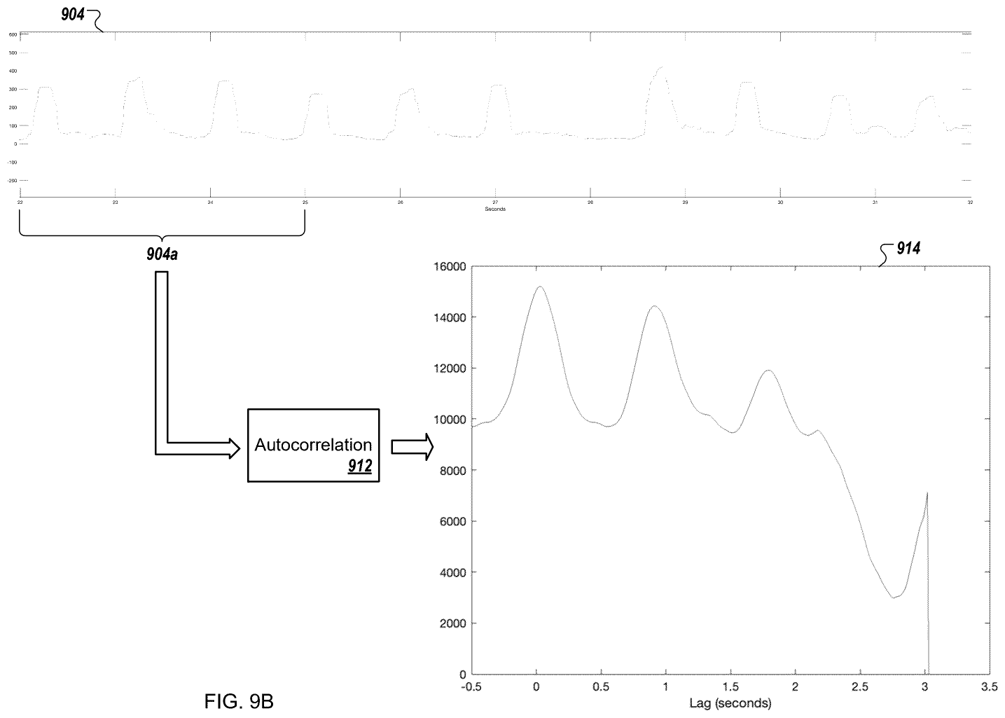

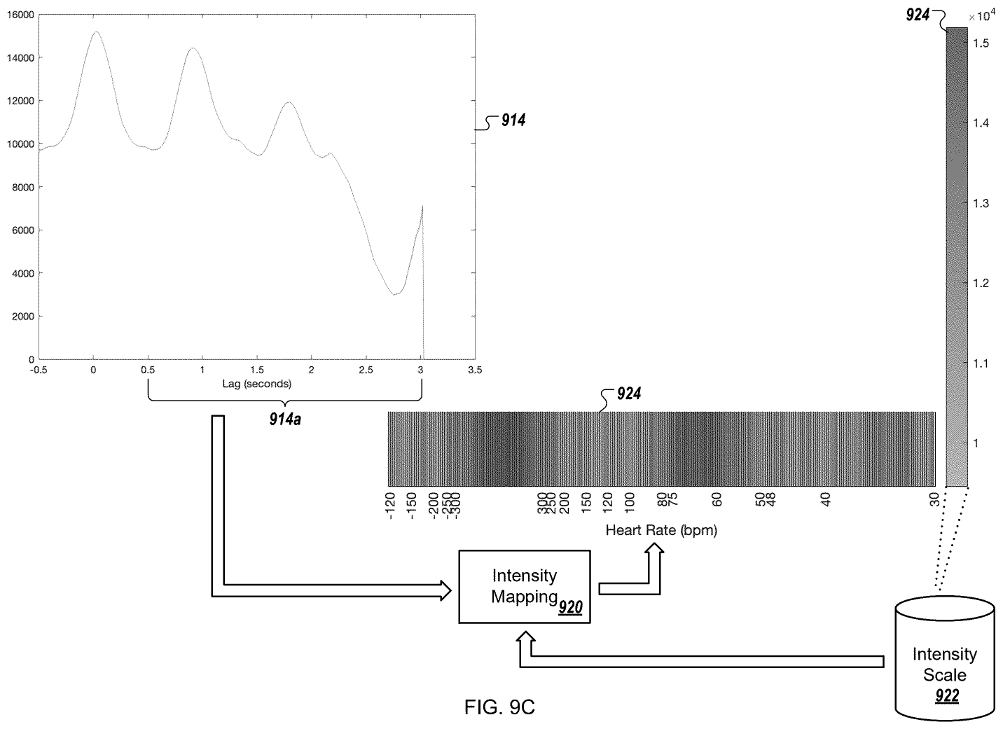

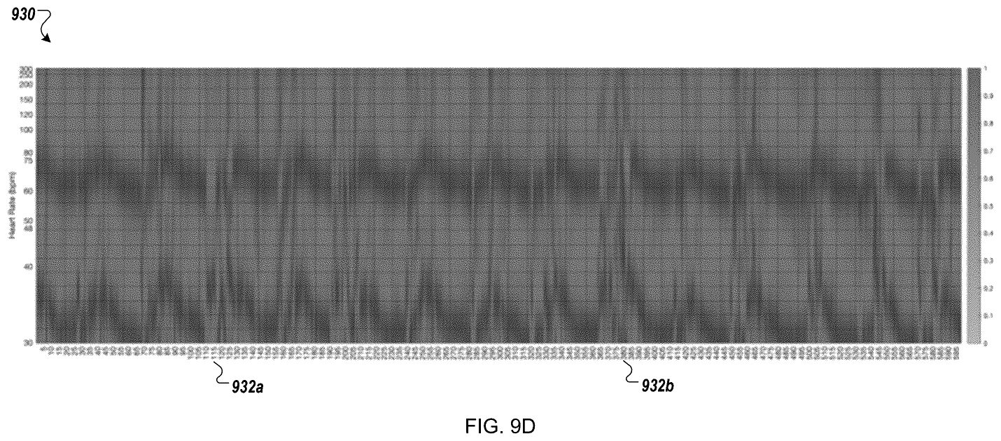

In another example, turning to FIG. 9B, heart sounds data, such as the refined heart sounds data 904, may be provided to an autocorrelation operation 910 to calculate autocorrelated heart sounds measurements 914.

Returning to FIG. 5A, in some implementations, if high-pass filtering is desired (508), each set of time lag measurements is filtered to intensify the local peaks therein (510). The filtering, for example, may be accomplished as described in relation to the filtering engine 314 of FIG. 3A.

Turning to FIG. 6B, a block diagram of an illustrative example of data filtering 620 is shown. A high-pass filter operation 622 may be applied to the autocorrelated data measurements 608 of FIG. 6A to accentuate a set of peaks 628a-e of the autocorrelated data measurements 608, resulting in filtered, autocorrelated data measurements 624. As illustrated, the filtered, autocorrelated data measurements 624 range in value, according to y-axis degree of similarity 626, from about −0.05 to about 0.33. The filter, in the illustrative example, has a cutoff frequency of 0.5 pi radians. As can be seen in the filtered-autocorrelated data measurements 624, a set of peaks 630a-e each have a greater data value than any non-peak data point, unlike the peaks 628a-e of the autocorrelated data measurements 608.

Returning to FIG. 5A, in some implementations, if negative lag filtering is desired (512), each set of time lag measurements is filtered to remove any negative lags (514). For example, as illustrated in FIG. 6B, the lower range of the y-axis degree of similarity 626 of the filtered, autocorrelated data measurements 624 reaches to −0.05. The negative lags within the filtered, autocorrelated data measurements 624 may be removed through filtering. The filtering, for example, may be performed by the filtering engine 314 of FIG. 3A.

In some implementations, plot dimensions are determined for converting each time lag measurement to a respective visual pixel region (516). The plot dimensions, for example, may be determined according to the one or more pixel scale parameters 322 of FIG. 3A. The data value to pixel mapping engine 320 of FIG. 3A, for example, may determine the plot dimensions.

Turning to FIG. 5B, in some implementations where the raw physiological data represents cardiac measurements (518), each time lag measurement of the set of time lag measurements is mathematically converted to a heart rate (520). The conversion, for example, may be performed by multiplying each time lag measurement M_{TL} by 60 and dividing by time t: M_{TL}*60/t.

In some implementations, at least a portion of the set of measurements for the first time period is accessed (522). For example, the filtered, time-lagged measurements 624 of FIG. 6B, corresponding to the cardiac data measurements 602 of time period 604a (as shown in FIG. 6A), may be accessed. As illustrated in FIG. 6B, the filtered, time-lagged measurements 624, as well as the time-lagged measurements 608, include duplicate measurements-a first half of the measurements of the time-lagged data set 608, 624 are a mirror image of the second half of the measurements of the time-lagged data set 608, 624. To reduce complexity of the resultant graphical image, in some embodiments, the time-lagged measurement data set used in creating the visual representation of the cardiac data set is truncated to only capture a portion of the data, such as only half of the “mirror” copy. The portion of the data set may be selected from various starting points, such as, in some examples, measurements from a first half of the overall time (e.g., second −2 to second 0), measurements from a second half of the overall time (e.g., second 0 to second 2), measurements from a first unique peak to a last unique peak (e.g., discarding duplicate peaks, discarding duplicate peaks plus a section of non-duplicate data that lacks a duplicate peak, discarding a peak representing a harmonic of an included peak, etc.).

In some implementations, each time lag measurement of the accessed time-lagged measurements may be converted to an image plot of a corresponding pixel on a pixel scale, producing an image having side-by-side visual regions of pixels (524). The time lag measurements may be converted, for example, as described in relation to the data value to pixel mapping engine 320 of FIG. 3A, according to the one or more pixel scale parameters 322.

Turning to FIG. 6C, a block diagram illustrates an example visual data conversion 640 of time-lagged measurements. As shown, a two-second window 642 of the filtered, correlated data measurements 624 (e.g., ranging from −0.5 seconds to 1.5 seconds on the time axis 612) is provided to an intensity mapping operation 644. The two-second window 642, for example, as a portion of the set of time-lagged data measurements, as described in relation to operation 522 of FIG. 5B. The intensity mapping operation 644, for example, may convert each time-lagged data measurement of the filtered, correlated measurements 624 within the two-second window 642 to a corresponding pixel value (e.g., color, hue, tone, and/or shade) according to an intensity scale 646. The intensity scale 646, as illustrated in a visual scale 648 ranging from aqua blue to fuchsia, may be applied to each of the measurements of the two-second window 642 to produce an image 650 having side-by-side visual regions of pixels (e.g., narrow columns, as illustrated).

Turning to FIG. 9C, a block diagram illustrates an example visual data conversion of time-lagged heart sounds measurements 914 to produce an image 924 having side-by-side visual regions of pixels. An intensity mapping operation 920, for example, may convert each time-lagged heart sounds data measurement of the time-lagged heart measurements 914 to a corresponding pixel value (e.g., color, hue, tone, and/or shade) according to an intensity scale 922. The intensity scale 922, as illustrated in a visual scale 924 ranging from aqua blue to fuchsia, may be applied to each of the measurements of the two and a half-second window 914a to produce an image 924 having side-by-side visual regions of pixels (e.g., narrow columns, as illustrated).

Returning to FIG. 5B, in some implementations, for each additional set of time measurements (526), the set of time lag measurements for the next time period is accessed (528) and converted (524). As discussed previously, each set of time measurements may correspond to a contiguous or partially overlapping segment of time of the original raw physiological (respiratory and/or cardiac monitoring) data.

In some implementations, after conversion of each contiguous set of time lag measurements (528), it is determined whether to adjust the orientation of the images (530). The orientation may be adjusted, for example, based on desired end user presentation. The presentation, for example, could include time moving from left to right, time moving from top to bottom, time moving from bottom to top, or time moving from right to left of the user display. Turning to FIG. 6D, for example, a block diagram of an example orientation adjustment 660 illustrates that pixel values, initially represented in the image 650 of FIG. 6C as a series of columns stacked side-by-side over time (e.g., from left to right), may be rotated to adjust orientation. As illustrated, an orientation adjustment 662 applied to the image 650 has rotated the image 650 to the right by 180 degrees to produce a column of visual pixel values, each visual pixel value represented by a short wide rectangle. Although illustrated as including a heart rate scale (e.g., the “x-axis” of the image 650, rotating to become the “y-axis” of the image 664), typically the image at this point would not include additional information.

Returning to FIG. 5B, in some implementations, the images of the time periods are arranged consecutively side-by-side to produce a visual intensity map of the physiological data (534). The graphical visualization building engine 326 of FIG. 3A, for example, may arrange the images of the time periods side-by-side according to the visual rendering parameter(s) 328.

Turning to FIG. 6E, a block diagram of an example visual arrangement 682 of the images of the time periods is illustrated. The visual image corresponding to each time period is represented as a separate column corresponding to a single second of time, where the data used to produce the individual seconds of graphical data, as illustrated in a mapping between the original cardiac monitoring data 602 and the visual arrangement 682, include overlapping two-second windows of time 664. For example, the data measurements of a first two-second time period 664a of the cardiac data 602 was used to produce pixel values for a first one-second visual column 682a of the visual representation; the data measurements of a second two-second time period 664b of the cardiac data 602 was used to produce pixel values for a second one-second visual column 682b of the visual representation; and the data measurements of a fifth two-second time period 664e of the cardiac data 602 was used to produce pixel values for a fifth one-second visual column 682e of the visual representation.

Turning to FIG. 9D, an example visual arrangement 930 of the images of consecutive time periods of heart sounds signals is illustrated. The visual arrangement 930 includes a set of periodic waves of fuchsia running horizontally across a generally teal background. At certain points of time, such as blocks of time including 115 seconds 932a and 380 seconds 932b, vertical disruptions can be discerned within the otherwise substantially similar, periodic waves.

Returning to the method 500 of FIG. 5A and FIG. 5B, although described in relation to a particular set of operations, in other embodiments, the method 500 includes more or fewer operations. For example, rather than producing separate images (524) for each set of time measurements, in some embodiments, a logical format of pixel values (e.g., a vector or matrix) can be used to produce a single image according to the mapped pixel values (e.g., as described in relation to FIG. 3A). In another example, rather than correlating (506) and then filtering (508-514) the monitoring data, an unbiased estimate of autocorrelation may be calculated. In another example, turning to FIG. 9A, a set of raw heart sounds data signals 900 may be reduced in complexity (e.g., simplified or smoothed) to exaggerate peaks in the data, for example using a moving standard deviation operation 902, thereby producing refined data signals 904, prior to correlating (506). Additionally, although the operations of the method 500 are present in a particular order, in other embodiments, certain operations may be performed in a different order and/or concurrently. For example, individual images may be rotated (532) after production (524). Other modifications of the method 500 are possible.

In some implementations, an unbiased estimate of autocorrelation is calculated using the following equation:

$${\hat{R}}_{xy , unbiased} ( m ) = \frac{1}{N - \vert m \vert} {\hat{R}}_{x y} ( m )$$

where m is the lag and N is signal length; and

$${\hat{R}}_{xy} ( m ) = \{ \begin{matrix}{\sum }_{n = 0}^{N - m - 1} {x}_{n + m {ϒ}_{n}^{*}} , & m \geq 0 , \\ {\hat{R}}_{yx}^{*} ( - m ) , & m < 0.\end{matrix}$$

In the above, equation, x and y are the input signal.

FIG. 7 illustrates an example component-level view of a medical device controller 700 included in, for example, a wearable medical device. As further shown in FIG. 7, the therapy delivery circuitry 702 can be coupled to one or more electrodes 720 configured to provide therapy to the patient. For example, the therapy delivery circuitry 702 can include, or be operably connected to, circuitry components that are configured to generate and provide an electrical therapeutic shock. The circuitry components can include, for example, resistors, capacitors, relays and/or switches, electrical bridges such as an h-bridge (e.g., including a number of insulated gate bipolar transistors or IGBTs), voltage and/or current measuring components, and other similar circuitry components arranged and connected such that the circuitry components work in concert with the therapy delivery circuitry and under control of one or more processors (e.g., processor 718) to provide, for example, at least one therapeutic shock to the patient including one or more pacing, cardioversion, or defibrillation therapeutic pulses.

Pacing pulses can be used to treat cardiac arrhythmia conditions such as bradycardia (e.g., less than 30 beats per minute) and tachycardia (e.g., more than 150 beats per minute) using, for example, fixed rate pacing, demand pacing, anti-tachycardia pacing, and the like. Defibrillation shocks can be used to treat ventricular tachycardia and/or ventricular fibrillation.

For example, each defibrillation shock can deliver between 60 to 180 joules of energy. In some implementations, the defibrillating shock can be a biphasic truncated exponential waveform, whereby the signal can switch between a positive and a negative portion (e.g., charge directions). This type of waveform can be effective at defibrillating patients at lower energy levels when compared to other types of defibrillation shocks (e.g., such as monophasic shocks). For example, an amplitude and a width of the two phases of the energy waveform can be automatically adjusted to deliver a precise energy amount (e.g., 150 joules) regardless of the patient's body impedance. The therapy delivery circuitry 702 can be configured to perform the switching and pulse delivery operations, e.g., under control of the processor 718. As the energy is delivered to the patient, the amount of energy being delivered can be tracked. For example, the amount of energy can be kept to a predetermined constant value even as the pulse waveform is dynamically controlled based on factors such as the patient's body impedance which the pulse is being delivered.

In certain examples, the therapy delivery circuitry 702 can be configured to deliver a set of cardioversion pulses to correct, for example, an improperly beating heart. When compared to defibrillation as described above, cardioversion typically includes a less powerful shock that is delivered at a certain frequency to mimic a heart's normal rhythm.

A data storage region 704 can include one or more of non-transitory (non-volatile) computer-readable media, such as flash memory, solid state memory, magnetic memory, optical memory, cache memory, combinations thereof, and others. The data storage 704 can be configured to store executable instructions and data used for operation of the medical device controller 700. In certain examples, the data storage 704 can include executable instructions that, when executed, are configured to cause the processor 718 to perform one or more operations. In some examples, the data storage 704 can be configured to store information such as ECG data as received from, for example, a sensing electrode interface 712.

In some embodiments, a network interface 706 can facilitate the communication of information between the medical device controller 700 and one or more other devices or entities over a communications network. For example, where the medical device controller 700 is included in an ambulatory medical device, the network interface 706 can be configured to communicate with a remote computing device such as a remote server or other similar computing device. In further embodiments, the remote computing device can be part of a remote data analytics system 732. The network interface 706 can include communications circuitry for transmitting data in accordance with a Bluetooth® or Zigbee® wireless standard for exchanging such data over short distances to an intermediary device 734. In some examples, the intermediary device 734 can be configured as a base station, a “hotspot” device, a smartphone, a tablet, a portable computing device, and/or other devices in proximity of the wearable medical device including the medical device controller 700. The intermediary device(s) 734 may in turn communicate the data to a remote server over a broadband cellular network communications link, such as the data analytics system 732. The communications link may implement broadband cellular technology (e.g., 2.5G, 2.75G, 3G, 4G, 5G cellular standards) and/or Long-Term Evolution (LTE) technology or GSM/EDGE and UMTS/HSPA technologies for high-speed wireless communication. In some implementations, the intermediary device(s) 734 may communicate with a remote server over a Wi-Fi® communications link based on the IEEE 802.11 standard.

In certain embodiments, a user interface 708 can include one or more physical interface devices such as input devices, output devices, and combination input/output devices and a software stack configured to drive operation of the devices. These user interface elements can render visual (e.g., LEDs 742), audio (e.g., speaker 740), and/or tactile content. Thus, the user interface 708 can receive input (e.g., via one or more control buttons 744) or provide output, thereby enabling a user to interact with the medical device controller 700.

The medical device controller 700, in some embodiments, includes at least one power source (e.g., rechargeable battery) 710 configured to provide power to one or more components integrated in the medical device controller 700. The battery 710 can include a rechargeable multi-cell battery pack. In one example implementation, the battery 710 can include three or more 2200 mAh lithium-ion cells that provide electrical power to the other device components within the medical device controller 700. For example, the battery 710 can provide its power output in a range of between 20 mA to 700 mA (e.g., 40 mA) output and can support 24 hours, 48 hours, 72 hours, or more, of runtime between charges. In certain implementations, the battery capacity, runtime, and type (e.g., lithium ion, nickel-cadmium, or nickel-metal hydride) can be changed to best fit the specific application of the medical device controller 700.

A sensor interface 712, in some embodiments, includes physiological signal circuitry that is coupled to one or more sensors configured to monitor one or more physiological parameters of the patient. As shown, the sensors 722, 724, 726, 730 can be coupled to the medical device controller 700 via a wired or wireless connection. The sensors can include one or more ECG sensing electrodes 722 and non-ECG physiological sensors such as a vibration sensor 724, tissue fluid monitor(s) 726 (e.g., based on ultra-wide band RF devices), and motion sensor(s) 730 (e.g., accelerometers, gyroscopes, and/or magnetometers). In some implementations, the sensors can include a number of conventional ECG sensing electrodes 722 in addition to digital sensing electrodes 722.

The sensing electrodes 722 can be configured to monitor a patient's ECG information. For example, by design, the digital sensing electrodes 722 can include skin-contacting electrode surfaces that may be deemed polarizable or non-polarizable depending on a variety of factors including the metals and/or coatings used in constructing the electrode surface. All such electrodes can be used with the principles, techniques, devices and systems described herein. For example, the electrode surfaces can be based on stainless steel, noble metals such as platinum, or Ag—AgCl.

In some examples, the electrodes 722 can be used with an electrolytic gel dispersed between the electrode surface and the patient's skin. In certain implementations, the electrodes 722 can be dry electrodes that do not need an electrolytic material. As an example, such a dry electrode can be based on tantalum metal and having a tantalum pentoxide coating as is described above. Such dry electrodes can be more comfortable for long term monitoring applications.

The vibration sensor(s) 724, in some implementations, can be configured to detect cardiac or pulmonary vibration information. For example, the vibration sensor(s) 724 can detect a patient's heart valve vibration information. For example, the vibration sensor(s) 724 can be configured to detect cardio-vibrational signal values including any one or all of S1, S2, S3, and S4. From these cardio-vibrational signal values or heart vibration values, certain heart vibration metrics may be calculated, including any one or more of electromechanical activation time (EMAT), average EMAT, percentage of EMAT (% EMAT), systolic dysfunction index (SDI), and left ventricular systolic time (LVST). The vibration sensor(s) 724 can also be configured to detect heart wall motion, for instance, by placement of the sensor in the region of the apical beat. The vibration sensor(s) 724 can include a vibrational sensor configured to detect vibrations from a subject's cardiac and pulmonary system and provide an output signal responsive to the detected vibrations of a targeted organ, for example, being able to detect vibrations generated in the trachea or lungs due to the flow of air during breathing. In certain implementations, additional physiological information can be determined from pulmonary-vibrational signals such as, for example, lung vibration characteristics based on pulmonary vibrations produced within the lungs (e.g., stridor, crackle, etc.). The vibration sensor(s) 724 can also include a multi-channel accelerometer, for example, a three-channel accelerometer configured to sense movement in each of three orthogonal axes such that patient movement/body position can be detected and correlated to detected cardio-vibrations information. The vibration sensor(s) 724 can transmit information descriptive of the cardio-vibrations information to the sensor interface 712 for subsequent analysis.

The tissue fluid monitor(s) 726 can use radio frequency (RF) based techniques to assess fluid levels and accumulation in a patient's body tissue. For example, the tissue fluid monitor(s) 726 can be configured to measure fluid content in the lungs, typically for diagnosis and follow-up of pulmonary edema or lung congestion in heart failure patients. The tissue fluid monitor(s) 726 can include one or more antennas configured to direct RF waves through a patient's tissue and measure output RF signals in response to the waves that have passed through the tissue. In certain implementations, the output RF signals include parameters indicative of a fluid level in the patient's tissue. The tissue fluid monitor(s) 726 can transmit information descriptive of the tissue fluid levels to the sensor interface 712 for subsequent analysis.

In certain implementations, a cardiac event detector 716 can be configured to monitor a patient's ECG signal for an occurrence of a cardiac event such as an arrhythmia or other similar cardiac event. The cardiac event detector 716 can be configured to operate in concert with the processor 718 to execute one or more methods that process received ECG signals from, for example, the sensing electrodes 722 and determine the likelihood that a patient is experiencing a cardiac event. The cardiac event detector 716 can be implemented using hardware or a combination of hardware and software. For instance, in some examples, cardiac event detector 716 can be implemented as a software component that is stored within the data storage 704 and executed by the processor 718. In this example, the instructions included in the cardiac event detector 716 can cause the processor 718 to perform one or more methods for analyzing a received ECG signal to determine whether an adverse cardiac event is occurring. In other examples, the cardiac event detector 716 can be an application-specific integrated circuit (ASIC) that is coupled to the processor 718 and configured to monitor ECG signals for adverse cardiac event occurrences. Thus, examples of the cardiac event detector 716 are not limited to a particular hardware or software implementation.

In some implementations, the processor 718 includes one or more processors (or one or more processor cores) that each are configured to perform a series of instructions that result in manipulated data and/or control the operation of the other components of the medical device controller 700. In some implementations, when executing a specific process (e.g., cardiac monitoring), the processor 718 can be configured to make specific logic-based determinations based on input data received and be further configured to provide one or more outputs that can be used to control or otherwise inform subsequent processing to be carried out by the processor 718 and/or other processors or circuitry with which processor 718 is communicatively coupled. Thus, the processor 718 reacts to specific input stimulus in a specific way and generates a corresponding output based on that input stimulus. In some example cases, the processor 718 can proceed through a sequence of logical transitions in which various internal register states and/or other bit cell states internal or external to the processor 718 can be set to logic high or logic low. As referred to herein, the processor 718 can be configured to execute a function where software is stored in a non-volatile computer-readable data store coupled to the processor 718, the software being configured to cause the processor 718 to proceed through a sequence of various logic decisions that result in the function being executed. The various components that are described herein as being executable by the processor 718 can be implemented in various forms of specialized hardware, software, or a combination thereof. For example, the processor 718 can be a digital signal processor (DSP) such as a 24-bit DSP. The processor 718 can be a multi-core processor, e.g., having two or more processing cores. The processor 718 can be an Advanced RISC Machine (ARM) processor such as a 32-bit ARM processor or a 64-bit ARM processor. The processor 718 can execute an embedded operating system, and include services provided by the operating system that can be used for file system manipulation, display & audio generation, basic networking, firewalling, data encryption and communications.

As noted above, an ambulatory medical device such as a WCD can be designed to include a digital front-end where analog signals sensed by skin-contacting electrode surfaces of a set of digital sensing electrodes are converted to digital signals for processing. Typical ambulatory medical devices with analog front-end configurations use circuitry to accommodate a signal from a high source impedance from the sensing electrode (e.g., having an internal impedance range from approximately 10 Kiloohms to one or more Megaohms). This high source impedance signal is processed and transmitted to a monitoring device such as processor 718 of the controller 700 as described above for further processing. In certain implementations, the monitoring device, or another similar processor such as a microprocessor or another dedicated processor operably coupled to the sensing electrodes, can be configured to receive a common noise signal from each of the sensing electrodes, sum the common noise signals, invert the summed common noise signals and feed the inverted signal back into the patient as a driven ground using, for example, a driven right leg circuit to cancel out common mode signals.

FIG. 8A illustrates an example medical device 800 that is external, ambulatory, and wearable by a patient 802, and configured to implement one or more configurations described herein. For example, the medical device 800 can be a non-invasive medical device configured to be located substantially external to the patient. Such a medical device 800 can be, for example, an ambulatory medical device that is capable of and designed for moving with the patient as the patient goes about his or her daily routine. For example, the medical device 800 as described herein can be bodily-attached to the patient such as the Life Vest® wearable cardioverter defibrillator available from ZOLL® Medical Corporation. Such wearable defibrillators (wearable cardiac medical devices or wearable cardiac monitoring devices) typically are worn nearly continuously or substantially continuously for two to three months at a time. During the period of time in which they are worn by the patient, the wearable defibrillator can be configured to continuously or substantially continuously monitor the vital signs of the patient and, upon determination that treatment is required, can be configured to deliver one or more therapeutic electrical pulses to the patient. For example, such therapeutic shocks can be pacing, defibrillation, or transcutaneous electrical nerve stimulation (TENS) pulses.

The medical device 800 can include one or more of the following: a garment 810, one or more ECG sensing electrodes 812, one or more non-ECG physiological sensors such as the sensors 724, 726, 730 described in relation to FIG. 7, one or more therapy electrodes 814a and 814b (collectively referred to herein as therapy electrodes 814), a medical device controller 820 (e.g., controller 700 as described above in the discussion of FIG. 7), a connection pod 830, a patient interface pod 840, a belt 850, or any combination of these. In some examples, at least some of the components of the medical device 800 can be configured to be affixed to the garment 810 (or in some examples, permanently integrated into the garment 810), which can be worn about the patient's torso. In some implementations, at least a portion of the components of the medical device 800 can be configured to be in wireless communication with other components of the medical device 800. For example, the patient interface pod 840 may be arranged as a remote-control interface for use by the patient and in wireless communication with the medical device controller 820.

The medical device controller 820 can be operatively coupled to the sensing electrodes 812, which can be affixed to the garment 810, e.g., assembled into the garment 810 or removably attached to the garment, for example using hook and loop fasteners, snaps, and/or Velcro. In some implementations, the sensing electrodes 812 can be permanently integrated into the garment 810. The medical device controller 820 can be operatively coupled to the therapy electrodes 814. For example, the therapy electrodes 814 can also be assembled into the garment 810, or, in some implementations, the therapy electrodes 814 can be permanently integrated into the garment 810. In an example, the medical device controller 820 includes a patient user interface 860 to allow a patient interface with the externally-worn device. For example, the patient can use the patient user interface 860 to respond to activity related questions, prompts, and surveys as described herein.

Component configurations other than those shown in FIG. 8A are possible. For example, the sensing electrodes 812 can be configured to be attached at various positions about the body of the patient 802. The sensing electrodes 812 can be operatively coupled to the medical device controller 820 through the connection pod 830. In some implementations, the sensing electrodes 812 can be adhesively attached to the patient 802. In some implementations, the sensing electrodes 812 and at least one of the therapy electrodes 814 can be included on a single integrated patch and adhesively applied to the patient's body.

The sensing electrodes 812 can be configured to detect one or more cardiac signals. Examples of such signals include ECG signals and/or other sensed cardiac physiological signals from the patient. In certain examples, as described herein, the non-ECG physiological sensors 813 such as accelerometers, vibrational sensors, RF-based sensors, and other measuring devices for recording additional non-ECG physiological parameters. For example, as described above, the non-ECG physiological sensors may be configured to detect other types of patient physiological parameters and acoustic signals, such as tissue fluid levels, cardio-vibrations, lung vibrations, respiration vibrations, and/or patient movement, etc.

In some examples, the therapy electrodes 814 can also be configured to include sensors configured to detect ECG signals as well as other physiological signals of the patient. The connection pod 830 can, in some examples, include a signal processor configured to amplify, filter, and digitize these cardiac signals prior to transmitting the cardiac signals to the medical device controller 820. One or more of the therapy electrodes 814 can be configured to deliver one or more therapeutic defibrillating shocks to the body of the patient 802 when the medical device 800 determines that such treatment is warranted based on the signals detected by the sensing electrodes 812 and processed by the medical device controller 820. Example therapy electrodes 814 can include metal electrodes such as stainless-steel electrodes that include one or more conductive gel deployment devices configured to deliver conductive gel to the metal electrode prior to delivery of a therapeutic shock.

In some examples, the medical device 800 can further includes one or more motion sensors such as accelerometers 862. As shown in FIG. 8A, in some examples an accelerometer 862 can be integrated into one or more of a sensing electrode 812, a therapy electrode 814, the medical device controller 820, and various other components of the medical device 800.

In some implementations, medical devices as described herein can be configured to switch between a therapeutic medical device and a monitoring medical device that is configured to only monitor a patient (e.g., not provide or perform any therapeutic functions). For example, therapeutic components such as the therapy electrodes 814 and associated circuitry can be optionally decoupled from (or coupled to) or switched out of (or switched in to) the medical device 800a. For example, a medical device can have optional therapeutic elements (e.g., defibrillation and/or pacing electrodes, components, and associated circuitry) that are configured to operate in a therapeutic mode. The optional therapeutic elements can be physically decoupled from the medical device to convert the therapeutic medical device into a monitoring medical device for a specific use (e.g., for operating in a monitoring-only mode) or a patient. Alternatively, the optional therapeutic elements can be deactivated (e.g., via a physical or a software switch), essentially rendering the therapeutic medical device as a monitoring medical device for a specific physiologic purpose or a particular patient. As an example of a software switch, an authorized person can access a protected user interface of the medical device and select a preconfigured option or perform some other user action via the user interface to deactivate the therapeutic elements of the medical device.

FIG. 8B illustrates a hospital wearable defibrillator 800b that is external, ambulatory, and wearable by the patient 802. Hospital wearable defibrillator 800b can be configured in some implementations to provide pacing therapy, e.g., to treat bradycardia, tachycardia, and asystole conditions. The hospital wearable defibrillator 800b can include one or more ECG sensing electrodes 812, one or more therapy electrodes 814, a medical device controller 820 and a connection pod 830. For example, each of these components can be structured and function as like number components of the medical device 800a of FIG. 8A. For example, the electrodes 812a-1112c, 814a, 814b can include disposable adhesive electrodes. For example, the electrodes 812a, 814a, 814b can include sensing and therapy components disposed on separate sensing and therapy electrode adhesive patches.

In some implementations, both sensing and therapy components can be integrated and disposed on a same electrode adhesive patch that is then attached to the patient. For example, the front adhesively attachable therapy electrode 814a attaches to the front of the patient's torso to deliver pacing or defibrillating therapy. Similarly, the back adhesively attachable therapy electrode 814b attaches to the back of the patient's torso. In an example scenario, at least three ECG adhesively attachable sensing electrodes 812a-1112c can be attached to at least above the patient's chest near the right arm, above the patient's chest near the left arm, and towards the bottom of the patient's chest in a manner prescribed by a trained professional.

A patient being monitored by a hospital wearable defibrillator and/or pacing device may be confined to a hospital bed or room for a significant amount of time (e.g., 75% or more of the patient's stay in the hospital). As a result, a user interface 860 can be configured to interact with a user other than the patient, e.g., a nurse, for device-related functions such as initial device baselining, setting and adjusting patient parameters, and changing the device batteries.

In some examples, the hospital wearable defibrillator 800b can further include one or more motion sensors such as accelerometers 862. As shown in FIG. 8B, in some examples an accelerometer 862 can be integrated into one or more of a sensing electrode 812a (e.g., integrated into the same patch as the sensing electrode), a therapy electrode 814a (e.g., integrated into the same patch as the therapy electrode), the medical device controller 820, the connection pod 830, and various other components of the hospital wearable defibrillator 800b.

In some implementations, an example of a therapeutic medical device that includes a digital front-end in accordance with the systems and methods described herein can include a short-term defibrillator and/or pacing device. For example, such a short-term device can be prescribed by a physician for patients presenting with syncope. A wearable defibrillator can be configured to monitor patients presenting with syncope by, e.g., analyzing the patient's physiological and cardiac activity for aberrant patterns that can indicate abnormal physiological function. For example, such aberrant patterns can occur prior to, during, or after the onset of syncope. In such an example implementation of the short-term wearable defibrillator, the electrode assembly can be adhesively attached to the patient's skin and have a similar configuration as the hospital wearable defibrillator described above in connection with FIG. 8B.

FIGS. 8C and 8D illustrate example wearable patient monitoring devices with no treatment or therapy functions. For example, such devices are configured to monitor one or more physiological parameters of a patient, e.g., for remotely monitoring and/or diagnosing a condition of the patient. For example, such physiological parameters can include a patient's ECG information, tissue (e.g., lung) fluid levels, cardio-vibrations (e.g., using accelerometers or microphones), and other related cardiac information. A cardiac monitoring device is a portable device that the patient can carry around as he or she goes about their daily routine.

Referring to FIG. 8C, an example wearable patient monitoring device 800c (e.g., an example heart failure management system (HFMS) device), that can include tissue fluid monitors 865 that use RF based techniques to assess fluid levels and accumulation in a patient's body tissue, is illustrated. Such tissue fluid monitors 865 can be configured to measure fluid content in the lungs, typically for diagnosis and follow-up of pulmonary edema or lung congestion in heart failure patients. The tissue fluid monitors 865 can include one or more antennas configured to direct RF waves through a patient's tissue and measure output RF signals in response to the waves that have passed through the tissue. In certain implementations, the output RF signals include parameters indicative of a fluid level in the patient's tissue. In examples, device 800c may be a cardiac monitoring device that also includes digital sensing electrodes 870a, 870b for sensing ECG activity of the patient. Device 800c can pre-process the ECG signals via one or more ECG processing and/or conditioning circuits such as an ADC, operational amplifiers, digital filters, signal amplifiers under control of a microprocessor. Device 800c can transmit information descriptive of the ECG activity and/or tissue fluid levels via a network interface to a remote server for analysis. Additionally, in certain implementations, the device 800c can include one or accelerometers 862 for measuring motion signals as described herein.

Referring to FIG. 8D, another example wearable cardiac monitoring device 800d can be attached to a patient 802 via at least three adhesive digital cardiac sensing electrodes 875a-c disposed about the patient's torso. Additionally, in certain implementations, the device 800d can include one or accelerometers (not illustrated) integrated into, for example, one or more of the digital sensing electrodes for measuring motion signals as described herein.

Cardiac devices 800c and 800d are used in cardiac monitoring and telemetry and/or continuous cardiac event monitoring applications, e.g., in patient populations reporting irregular cardiac symptoms and/or conditions. These devices can transmit information descriptive of the ECG activity and/or tissue fluid levels via a network interface to a remote server for analysis. Example cardiac conditions that can be monitored include atrial fibrillation (AF), bradycardia, tachycardia, atrio-ventricular block, Lown-Ganong-Levine syndrome, atrial flutter, sino-atrial node dysfunction, cerebral ischemia, pause(s), and/or heart palpitations. For example, such patients may be prescribed a cardiac monitoring for an extended period of time, e.g., 10 to 30 days, or more. In some ambulatory cardiac monitoring and/or telemetry applications, a portable cardiac monitoring device can be configured to substantially continuously monitor the patient for a cardiac anomaly, and when such an anomaly is detected, the monitor can automatically send data relating to the anomaly to a remote server. The remote server may be located within a 24-hour manned monitoring center, where the data is interpreted by qualified, cardiac-trained reviewers and/or HCPs, and feedback provided to the patient and/or a designated HCP via detailed periodic or event-triggered reports. In certain cardiac event monitoring applications, the cardiac monitoring device is configured to allow the patient to manually press a button on the cardiac monitoring device to report a symptom. For example, a patient can report symptoms such as a skipped beat, shortness of breath, light headedness, racing heart rate, fatigue, fainting, chest discomfort, weakness, dizziness, and/or giddiness. The cardiac monitoring device can record predetermined physiologic parameters of the patient (e.g., ECG information) for a predetermined amount of time (e.g., 1-30 minutes before and 1-30 minutes after a reported symptom). As noted above, the cardiac monitoring device can be configured to monitor physiologic parameters of the patient other than cardiac related parameters. For example, the cardiac monitoring device can be configured to monitor, for example, cardio-vibrational signals (e.g., using accelerometers or microphones), pulmonary-vibrational signals, breath vibrations, sleep related parameters (e.g., snoring, sleep apnea), tissue fluids, among others.

In some examples, the devices described herein (e.g., FIGS. 8A-11D) can communicate with a remote server via an intermediary or gateway device 880 such as that shown in FIG. 8D. For instance, devices such as shown in FIGS. 8A-D can be configured to include a network interface communications capability as described herein in reference to, for example, FIG. 10.

Additionally, the devices 800a-d described herein in relation to FIGS. 8A-11D can be configured to include one or more vibrational sensors as described herein for collecting signals for use in producing cardio-vibrational image matrices.

Reference has been made to illustrations representing methods and systems according to implementations of this disclosure. Aspects thereof may be implemented by computer program instructions. The computer program instructions may be executed by computing logic, such as hardware logic or software logic. These computer program instructions may be provided to a processor of a general-purpose computer, special purpose computer, or other programmable data processing apparatus and/or distributed processing systems having processing circuitry, such that the instructions, which execute via the processor of the computer or other programmable data processing apparatus, create means for implementing the functions/operations specified in the illustrations.

One or more processors can be utilized to implement various functions and/or algorithms described herein. Additionally, any functions and/or algorithms described herein can be performed upon one or more virtual processors. The virtual processors, for example, may be part of one or more physical computing systems such as a computer farm or a cloud drive.

Aspects of the present disclosure may be implemented by software logic, including machine readable instructions or commands for execution via processing circuitry. The software logic may also be referred to, in some examples, as machine readable code, software code, or programming instructions. The software logic, in certain embodiments, may be coded in runtime-executable commands and/or compiled as a machine-executable program or file. The software logic may be programmed in and/or compiled into a variety of coding languages or formats. The software logic may be stored to a non-transitory (non-volatile) computer readable medium and configured for execution on processing circuitry (e.g., one or more processors).

Aspects of the present disclosure may be implemented by hardware logic (where hardware logic naturally also includes any necessary signal wiring, memory elements and such), with such hardware logic able to operate without active software involvement beyond initial system configuration and any subsequent system reconfigurations (e.g., for different object schema dimensions). The hardware logic may be programmed into one or more processing devices. The hardware logic, for example, may be synthesized on a reprogrammable computing chip such as a field programmable gate array (FPGA) or other reconfigurable logic device. In an additional example, the hardware logic may be hard coded onto a custom microchip, such as an application-specific integrated circuit (ASIC). In some embodiments, software logic, for example stored as instructions to a non-transitory computer-readable medium such as a memory device, on-chip integrated memory unit, or other non-transitory computer-readable storage, may be used to perform at least portions of the herein described functionality.

Various aspects of the embodiments disclosed herein are performed on one or more computing devices, such as a laptop computer, tablet computer, mobile phone or other handheld computing device, or one or more servers. Such computing devices include processing circuitry embodied in one or more processors or logic chips, such as a central processing unit (CPU), graphics processing unit (GPU), field programmable gate array (FPGA), application-specific integrated circuit (ASIC), or programmable logic device (PLD). Further, the processing circuitry may be implemented as multiple processors cooperatively working in concert (e.g., in parallel) to perform the instructions of the inventive processes described above.

The process data and instructions used to perform various methods and algorithms derived herein may be stored in non-transitory (i.e., non-volatile) computer-readable medium or memory. The claimed advancements are not limited by the form of the computer-readable media on which the instructions of the inventive processes are stored. For example, the instructions may be stored on CDs, DVDs, in FLASH memory, RAM, ROM, PROM, EPROM, EEPROM, hard disk or any other information processing device with which the computing device communicates, such as a server or computer. The processing circuitry and stored instructions may enable the computing device to perform, in some examples, the method 200 of FIG. 2A and FIG. 2B, the process 300 of FIG. 3A, the process 350 of FIG. 3B, and/or the method 500 of FIG. 5A and FIG. 5B.

These computer program instructions can direct a computing device or other programmable data processing apparatus to function in a particular manner, such that the instructions stored in the computer-readable medium produce an article of manufacture including instruction means which implement the function/operation specified in the illustrated process flows.

The computing device, in some embodiments, further includes a display controller for interfacing with a display, such as a built-in display or LCD monitor. A general purpose I/O interface of the computing device may interface with a keyboard, a hand-manipulated movement tracked I/O device (e.g., mouse, virtual reality glove, trackball, joystick, etc.), and/or touch screen panel or touch pad on or separate from the display.

Moreover, the present disclosure is not limited to the specific circuit elements described herein, nor is the present disclosure limited to the specific sizing and classification of these elements. For example, the skilled artisan will appreciate that the circuitry described herein may be adapted based on changes in battery sizing and chemistry or based on the requirements of the intended back-up load to be powered.

Although provided for context, in other implementations, methods and logic flows described herein may be performed on modules or hardware not identical to those described. Accordingly, other implementations are within the scope that may be claimed.

While certain embodiments have been described, these embodiments have been presented by way of example only, and are not intended to limit the scope of the present disclosures. Indeed, the novel methods, apparatuses and systems described herein can be embodied in a variety of other forms; furthermore, various omissions, substitutions and changes in the form of the methods, apparatuses and systems described herein can be made without departing from the spirit of the present disclosures. The accompanying claims and their equivalents are intended to cover such forms or modifications as would fall within the scope and spirit of the present disclosures.

1.-42. (canceled)

43. A cardiac data review system for identifying potential arrhythmias from a wearable medical device, the system comprising:
a communication interface configured to receive full-resolution electrocardiogram (ECG) data captured by a wearable medical device monitoring a subject over an extended period of time; and
computing logic comprising at least one of i) hardware logic programmed into one or more processing devices or ii) software logic stored to a non-volatile computer readable medium and configured for executing on one or more processors, the computing logic being operably coupled to the communication interface and a display, the computing logic being configured to:
segment the full-resolution ECG data into a plurality of contiguous, fixed-length time periods;
for each respective time period of the plurality of time periods:
perform an autocorrelation on a respective portion of the ECG data corresponding to the respective time period to generate a set of correlation values; and
convert the set of correlation values into a corresponding series of pixels, wherein each pixel has a value determined by a magnitude of a corresponding correlation value; and
generate for presentation on the display a visual grid by arranging each series of pixels as a respective column in chronological order, the visual grid configured to present a stable cardiac rhythm as a discernible horizontal pattern and an arrhythmic event as a visual disruption to the discernible horizontal pattern.

44. The system of claim 43, wherein the wearable medical device is a wearable cardioverter defibrillator.

45. The system of claim 43, wherein the visual grid is a heat map, and the value of each pixel is represented by a specific color or intensity from a predefined color scale.

46. The system of claim 43, wherein the fixed-length time period for segmentation is between 1 second and 5 seconds.

47. The system of claim 43, wherein the discernible horizontal pattern is indicative of a subject's heart rate, and the visual disruption is indicative of a cardiac condition selected from the group consisting of: supraventricular tachycardia (SVT), ventricular tachycardia, ventricular fibrillation, bradycardia, asystole, a heart pause, atrial fibrillation, and an ectopic beat.

48. The system of claim 43, wherein the computing logic is further configured to receive full-resolution cardio-vibrational sensor (CVG) data and generate a second visual grid based on the CVG data.

49. The system of claim 43, wherein the system is embodied in a monitoring station comprising the display and is located remotely from the subject.

50. A data visualization system for efficiently reviewing cardiac data, the system comprising:
a communication interface configured to receive, from a wearable cardioverter defibrillator, full-resolution ECG data spanning an extended time period;
a display;
at least one user input device; and
computing logic comprising at least one of i) hardware logic and/or ii) processing circuitry for executing software code stored to a non-volatile computer-readable medium as a plurality of instructions, the computing logic being operably coupled to the communication interface, the display, and the at least one user input device, the computing logic being configured to
generate a compact visual representation of the full-resolution ECG data by:
dividing the ECG data into a plurality of sequential time intervals;
for each time interval, calculating a set of time-lagged correlation values for the ECG data within that time interval; and
constructing an image by arranging a series of pixels corresponding to each set of time-lagged correlation values into a sequence of columns;
cause the display to render a first user interface presenting the compact visual representation, the compact visual representation comprising a visual pattern indicative of a baseline cardiac rhythm;
receive, via the at least one user input device, a user input selecting a region of the compact visual representation showing a disruption in the visual pattern; and
in response to receiving the user input, cause the display to render a second user interface presenting a raw waveform representation of the full-resolution ECG data corresponding to a timeframe of the selected region.

51. The system of claim 50, wherein the computing logic is configured to render the first user interface and the second user interface within a single graphical user interface on the display.

52. The system of claim 50, wherein the first user interface is configured for presentation to an ECG technician for triage and the second user interface is configured for presentation to a clinician for diagnosis.

53. The system of claim 50, wherein the user input comprises coordinates of a bounding box drawn around the disruption.

54. The system of claim 50, wherein the computing logic is further configured to, prior to constructing the image, apply a high-pass filter to the time-lagged correlation values to accentuate peaks therein.

55. The system of claim 50, wherein the extended time period is at least one hour.

56. The system of claim 50, wherein the compact visual representation is generated in near real-time as the full-resolution ECG data is received.

57. Computing logic comprising at least one of i) hardware logic programmed into one or more processing devices or ii) software logic stored to a non-volatile computer readable medium and configured for executing on one or more processors, wherein the computing logic, when executed by a cardiac monitoring system, cause the cardiac monitoring system to perform operations comprising:
accessing full-resolution physiological data captured by a wearable cardiac monitor over an extended period of time;
transforming the full-resolution physiological data into a visual grid, wherein the transformation comprises:
for each of a plurality of sequential time segments of the full-resolution physiological data, calculating a set of autocorrelation values; and
arranging pixel representations of each set of autocorrelation values into a series of columns to form the visual grid;
causing presentation of the visual grid on a display for review;
receiving an input identifying a visual disruption of a pattern within the visual grid indicative of a stable physiological rhythm; and
in response to receiving the input, initiating a clinical workflow action.

58. The computing logic of claim 57, wherein the clinical workflow action is generating an alert for presentation to a human reviewer.

59. The computing logic of claim 57, wherein the full-resolution physiological data comprises ECG data, the received input is indicative of ventricular fibrillation, and initiating the clinical workflow action comprises sending a command to a wearable cardioverter defibrillator to prepare a therapeutic shock.

60. The computing logic of claim 57, wherein the operations further comprise filtering the set of autocorrelation values according to a filtering parameter to visually accentuate the pattern or the visual disruption in the visual grid.

61. The computing logic of claim 57, wherein the full-resolution physiological data further comprises respiratory data, and the visual disruption is indicative of a sleep apnea event.

62. The computing logic of claim 57, wherein initiating the clinical workflow action comprises flagging a portion of the visual grid corresponding to the visual disruption and annotating the flagged portion with a label identifying a suspected cardiac condition.
